(19) United States
(12) Patent Application Publication
(10) Pub. No.: US 2023/0173040 A1
(43) Pub. Date: Jun. 8, 2023

(54) METHODS AND COMPOSITIONS FOR ATI DIGESTION

(71) Applicant: GLUTAGEN PTY LTD, Melbourne (AU)

(72) Inventors: Michelle Lisa COLGRAVE, Melbourne (AU); Angéla JUHÁSZ, Melbourne (AU); Gregory John TANNER, Melbourne (AU); Ezequiel PENA, Melbourne (AU)

(73) Assignee: GLUTAGEN PTY LTD, Melbourne (AU)

(21) Appl. No.: 17/998,428

(22) PCT Filed: May 12, 2021
(86) PCT No.: PCT/AU2021/050446

§ 371 Date: Nov. 10, 2022

(30) Foreign Application Priority Data
May 12, 2020 (AU) 2020901527

(51) Int. Cl. A61K 38/48; C12N 9/50; A23L 29/00

(52) U.S. Cl. CPC A61K 38/4873; C12Y 304/22002; C12N 9/63; A23L 29/06

(57) ABSTRACT

The present invention is generally related to methods of digesting α-amylase/trypsin inhibitors (ATI), and to the treatment and/or prophylaxis of an α-amylase/trypsin inhibitor (ATI) mediated conditions.

### FIELD OF THE INVENTION

The present invention is generally related to methods of digesting α-amylase/trypsin inhibitors (ATI), and to the treatment and/or prophylaxis of an α-amylase/trypsin inhibitor (ATI) mediated conditions.

### BACKGROUND

In the last decades, the implementation of novel agricultural practices contributed positively to the decrease of costs associated with large-scale production of wheat-based food. Consequently, the higher consumption of breads and pastas caused a predictable increase in hypersensitization to wheat. The most common of these disorders include baker's asthma, and immune reactions to wheat ingestion, such as celiac disease (CD), wheat allergy (WA), and non-celiac gluten/wheat sensitivity (NCGS or NCWS).

CD is triggered by gluten peptides that induce the adaptive immune response in predisposed individuals, resulting in the activation of T-cells, whereas IgE antibodies are induced by wheat proteins in WA, eventually stimulating the release of immune mediators.

NCGS is associated with innate immune activation, which is likely stimulated by wheat proteins. NCGS presents also extra-intestinal symptoms, such as confusion and headache, chronic fatigue, joint/muscle pain, and the exacerbation of pre-existing neurological, psychiatric, or (auto-)immune diseases.

Based on their structural, chemical and physical properties, wheat proteins are generally categorized as albumins and globulins (15% of total protein content), and gluten (85% of total protein content). Specifically, gluten consists of a complex mixture of monomeric gliadins and polymeric glutenins, whereas albumins and globulins comprise several families of proteins, such as the α-amylase/trypsin inhibitors (ATIs), 3-amylases, peroxidases, lipid transfer proteins, and serine proteases inhibitors. In the quest to identify wheat components effectively responsible for the initiation of innate immune response, ATIs were demonstrated as potent activators of myeloid cells. Specifically, ATIs directly engage TLR4-MD2-CD14 complex and activate both nuclear factor kappa B and interferon responsive factor 3 pathways, resulting in the up-regulation of maturation markers and the release of proinflammatory innate cytokines. The centrality of TLR4 system was further confirmed, as animal models deficient in TLR4 were protected from the intestinal and systemic immune responses upon oral challenge with ATIs15.

Compared to other protein constituents, ATIs represent a minor, but still significant part of total wheat proteins (2-4%), on average, an adult person being exposed up to 1 g of ATIs per day: in fact, ATIs are present and even enriched in commercial wheat-based food, and can escape proteolytic digestion by pepsin and trypsin, preserving the TLR4-activating ability after intestinal transit upon oral ingestion.

Structurally, wheat ATIs belong to a group of hydrolase-resistant proteins stabilized by inter-molecular disulfide bonds, and with high secondary structural homology. They can be further divided into three sub-groups constituted by monomeric and (non-covalently linked) dimeric and tetrameric forms. ATIs are found in the endosperm of plant seeds, where they represent part of the natural defence against parasites and insects, as well as regulatory molecules of starch metabolism during seed development and germination. Plants other than wheat, such as rye and barley also contain similar bi-functional inhibitors, but show only minimal or absent TLR4-activating activity.

Due to the in vivo TLR4 stimulatory activity and resistance to gastrointestinal proteolysis, this latter being attributable to the potent inhibitory activity toward diverse hydrolases, ATIs may exert a pathogenic role in inflammatory, metabolic and autoimmune diseases and in NCGS.

Currently no effective therapy is available to treat diseases mediated by ATIs save for imposing a strict avoidance diet on the patient. Foods that contain such proteins are major components of modern diets. Furthermore, the widespread use of ATI containing commodity products can lead to patients having difficulty ascertaining whether direct to consumer food products will pose a risk if ingested. Thus the number of food products containing sufficient ATIs to cause a severe reaction to the food is vast and often unpredictable. As such a diet strictly ATI exclusive is often difficult and inconvenient for a patient to implement.

In view of the serious, widespread nature ATI mediated diseases and the current difficulties associated with preventing and treating them, there remains a need to develop improved methods of treating, preventing or ameliorating the effects of these related conditions are needed.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In one aspect the present invention provides a method for digesting at least one alpha amylase trypsin inhibitor (ATI), the method comprising contacting an ATI with an effective dose of a composition comprising a proteolytic preparation comprising one or more Carica papaya endopeptidases.

In one aspect the present invention provides a method for digesting at least one alpha amylase trypsin inhibitor (ATI) in an ATI-containing foodstuff, the method comprises contacting the ATI-containing foodstuff with an effective dose of a composition comprising a proteolytic preparation comprising one or more Carica papaya endopeptidases.

In one embodiment the present invention provides a method as described herein, wherein the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5).

In another embodiment the present invention provides a method as described herein, wherein the alpha amylase trypsin inhibitor (ATI), is contacted with the proteolytic preparation comprising one or more Carica papaya endopeptidases under conditions sufficient to cause digestion of the ATI by hydrolysis.

In a further embodiment the present invention provides a method as described herein, wherein the at least one ATI is an ATI derived from a wheat, barley, rye or oat species.

In a further embodiment the present invention provides a method as described herein, wherein the at least one ATI is an ATI derived from a spelt, khorasan, emmer, einkorn, and triticale species.

In a further embodiment the present invention provides a method as described herein, wherein the at least one ATI is a wheat ATI.

In a further embodiment the present invention provides a method as described herein, wherein at least one epitope of the at least one ATI is digested.

In a further embodiment the present invention provides a method as described herein, wherein the at least one epitope is selected from;
a) an Alpha-amylase inhibitor precursor (CIII) (WMAI-1) B cell epitope selected from the group consisting of:

AASVPE
ADINNE
ALTGCR
AMVKLQ
AVLRDC
AYPDV
CQQLAD
CRAMVK
CVGSQV
CYGDWA
DCCQQL
ELGVRE
EVMKLT
GCRKEV
GDRAGV
GDWAAY
GKEVLP
GSQVPE
GVCYGD
GVREGK
INNEWC
KVPIPN
LPGCRK
LQCVGS
LRDCCQ
LRSVYQ
LSSMLR
LTAASV
MKLTAA
NEWCRC
PATGYK
PEAVLR
PEVCKV
PSGDRA
QLADIN
QVPEAV
RAGVCY
RCGDLS
REGKEV
RKEVMK
SGPWSW
SMLRSV
SVPEVC
SVYQEL
TGCRAM
TGYKVS
VCKVPI
VKLQCV
VSALTG
WAAYPD
WCRCGD
YKVSAL
YQELGV;

b) a putative alpha-amylase inhibitor B cell epitope selected from the group consisting of PWSWCD, SWCDPA, and SWCDPATGYKVSALTGCRAMV;
c) a peptide comprising an ATI epitope, said peptide selected from the group consisting of:

DCCQQLAHISEWCR
EHGAQEGQAGTGAFPR
CGALYSMLDSMYK
LQCNGSQVPEAVLR
LPIVVDASGDGAYVCK
LTAASITAVCR
SGPWMCYPGQAFQVPALPACRPL LR
DCCQQLADISEWCR
EHGVSEGQAGTGAFPSCR
SGPWMCYPGQAFQVPALPGCRPL LK
ECCQQLADISEWCR
LTAASITAVCK
SGPWMCYPGYAFK
VPALPGCRPVLK;

d) a CM16 and/or CM17 ATI epitope selected from the group consisting of:

IETPGSPYLAK
SDPNSSVLK
ELYDASQHCR
EYVAQQTCGVGIVGSPVSTEPGN TPR
TSDPNSGVLK
VLVTPGHCNVMTVHNTPYCLGLD I
IEMPGPPYLAK
NYVEEQACR
QECCEQLANIPQQCR
SRPDQSGLMELPGCPR
YFMGPK
EVQMDFVR;

e) a monomeric ATI epitope selected from the group consisting of:

EVLPGCR
CGDLSSMLR
DCCQQLADINNEWCR
VSALTGCR
SVYQELGVR
SHNSGPWSWCDPATGYK
LTAASVPEVCK
LQCVGSQVPEAVLR
VPIPNPSGDR
SVYQEIGVR;

f) a CM3 ATI epitope selected from the group consisting of:

LPEWMTSASIYSPGKPYLAK
EMQWDFVR
LYCCQELAEISQQCR
DLPGCPR
LLVAPGQCNLATIHNVR
DYVLQQTCGTFTPGSK
YFIALPVPSQPVDPR
SGNVGESGLIDLPGCPR
LPEWMTSASIFSPMKPYLAK
LYCCQELAEIPQQCR
QMQWDFVR
TDLLPHCR;

g) a CM Hagerman or CM1 ATI epitope selected from the group consisting of:

GPSLPMLVK
SDPNSSVLK;

and
h) a dimeric ATI epitope selected from the group consisting of:

EHGAQEGQAGTGAFPR
EFIAGIVGR.

In a further embodiment the present invention provides a method as described herein, wherein the proteolytic preparation is derived from Carica papaya oleoresin. In a further embodiment the present invention provides a method as described herein, wherein the proteolytic preparation is derived from Carica papaya latex. In a further embodiment the present invention provides a method as described herein, wherein the composition further comprises one or more excipients. In a further embodiment the present invention provides a method as described herein, wherein the method is performed in vitro or in vivo. In a further embodiment the present invention provides a method as described herein, wherein the composition is administered to a human.

In one aspect the present invention provides a method for the treatment and/or prevention of an alpha-amylase/trypsin inhibitor (ATI) mediated condition, comprising administering to a subject in need thereof an effective amount of a composition comprising a proteolytic preparation comprising one or more Carica papaya endopeptidases.

In one embodiment the present invention provides a method as described herein, wherein the condition is a wheat alpha-amylase/trypsin inhibitor (ATI) mediated condition

In another embodiment the present invention provides a method as described herein, wherein the alpha-amylase/trypsin inhibitor (ATI) mediated condition is wheat allergy, non-celiac gluten/wheat sensitivity, irritable bowel syndrome or baker's asthma.

In a further embodiment the present invention provides a method as described herein, wherein the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5).

In a further embodiment the present invention provides a method as described herein, wherein the proteolytic preparation comprises is derived from Carica papaya oleoresin. In a further embodiment the present invention provides a method as described herein, wherein the proteolytic preparation comprises is derived from Carica papaya latex. In a further embodiment the present invention provides a method as described herein, wherein the composition further comprises one or more pharmaceutically acceptable excipients. In a further embodiment the present invention provides a method as described herein, wherein the composition is administered orally. In a further embodiment the present invention provides a method as described herein, wherein the composition is enterically coated. In a further embodiment the present invention provides a method as described herein, wherein the subject is a human subject.

In one aspect the present invention provides a use of a composition comprising a proteolytic preparation comprising one or more Carica papaya endopeptidases in the manufacture of a medicament for the treatment and/or prophylaxis of an α-amylase/trypsin inhibitor (ATI) mediated condition.

In one embodiment the present invention provides a use as described herein, wherein the condition is a wheat alpha-amylase/trypsin inhibitor (ATI) mediated condition.

In another embodiment the present invention provides a use as described herein, wherein the α-amylase/trypsin inhibitor (ATI) mediated condition is wheat allergy, non-celiac gluten/wheat sensitivity, irritable bowel syndrome or baker's asthma. In a further embodiment the present invention provides a use as described herein, wherein the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5). In a further embodiment the present invention provides a use as described herein, wherein the proteolytic preparation is derived from Carica papaya oleoresin. In a further embodiment the present invention provides a use as described herein, wherein the proteolytic preparation is derived from Carica papaya latex. In a further embodiment the present invention provides a use as described herein, wherein the composition further comprises one or more pharmaceutically acceptable excipients. In a further embodiment the present invention provides a use as described herein, wherein the composition is administered orally. In a further embodiment the present invention provides a use as described herein, wherein the composition is enterically coated. In a further embodiment the present invention provides a use as described herein, wherein the subject is a human subject.

In one aspect the present invention provides a method for digesting an epitope of least one alpha amylase trypsin inhibitor (ATI), the method comprising contacting an ATI comprising the epitope with an effective dose of a composition comprising a proteolytic preparation comprising one or more Carica papaya endopeptidases.

In one embodiment the epitope is comprises one or more cysteine amino acids.

In one embodiment the present invention provides a method as described herein, wherein method according to claim 35 wherein the at least one epitope is selected from;
a) an Alpha-amylase inhibitor precursor (CIII) (WMAI-1) B cell epitope selected from the group consisting of:

AASVPE
ADINNE
ALTGCR
AMVKLQ
AVLRDC
AYPDV
CQQLAD
CRAMVK
CVGSQV
CYGDWA
DCCQQL
ELGVRE
EVMKLT
GCRKEV
GDRAGV
GDWAAY
GKEVLP
GSQVPE
GVCYGD
GVREGK
INNEWC
KVPIPN
LPGCRK
LQCVGS
LRDCCQ
LRSVYQ
LSSMLR
LTAASV
MKLTAA
NEWCRC
PATGYK
PEAVLR
PEVCKV
PSGDRA
QLADIN
QVPEAV
RAGVCY
RCGDLS
REGKEV
RKEVMK
SGPWSW
SMLRSV
SVPEVC
SVYQEL
TGCRAM
TGYKVS
VCKVPI
VKLQCV
VSALTG
WAAYPD
WCRCGD
YKVSAL
YQELGV;

b) a putative alpha-amylase inhibitor B cell epitope selected from the group consisting of PWSWCD, SWCDPA, and SWCDPATGYKVSALTGCRAMV;
c) a peptide comprising an ATI epitope, said peptide selected from the group consisting of:

DCCQQLAHISEWCR
EHGAQEGQAGTGAFPR
CGALYSMLDSMYK
LQCNGSQVPEAVLR
LPIVVDASGDGAYVCK
LTAASITAVCR
SGPWMCYPGQAFQVPALPACRPL LR
DCCQQLADISEWCR
EHGVSEGQAGTGAFPSCR
SGPWMCYPGQAFQVPALPGCRPL LK
ECCQQLADISEWCR
LTAASITAVCK
SGPWMCYPGYAFK
VPALPGCRPVLK;

d) a CM16 and/or CM17 ATI epitope selected from the group consisting of:

IETPGSPYLAK
SDPNSSVLK
ELYDASQHCR
EYVAQQTCGVGIVGSPVSTEPGN TPR
TSDPNSGVLK
VLVTPGHCNVMTVHNTPYCLGLD I
IEMPGPPYLAK
NYVEEQACR
QECCEQLANIPQQCR
SRPDQSGLMELPGCPR
YFMGPK
EVQMDFVR;

e) a monomeric ATI epitope selected from the group consisting of:

EVLPGCR
CGDLSSMLR
DCCQQLADINNEWCR
VSALTGCR
SVYQELGVR
SHNSGPWSWCDPATGYK
LTAASVPEVCK
LQCVGSQVPEAVLR
VPIPNPSGDR
SVYQEIGVR;

f) a CM3 ATI epitope selected from the group consisting of:

LPEWMTSASIYSPGKPYLAK
EMQWDFVR
LYCCQELAEISQQCR
DLPGCPR
LLVAPGQCNLATIHNVR
DYVLQQTCGTFTPGSK
YFIALPVPSQPVDPR
SGNVGESGLIDLPGCPR
LPEWMTSASIFSPMKPYLAK
LYCCQELAEIPQQCR
QMQWDFVR
TDLLPHCR;

g) a CM Hagerman or CM1 ATI epitope selected from the group consisting of:

GPSLPMLVK
SDPNSSVLK;

and
h) a dimeric ATI epitope selected from the group consisting of:

EHGAQEGQAGTGAFPR
EFIAGIVGR.

### BRIEF DESCRIPTION OF THE DRAWINGS

FIG. 1 illustrates the primary amino acid sequence of Carica papaya caricain (GenBank Accession No. X66060).

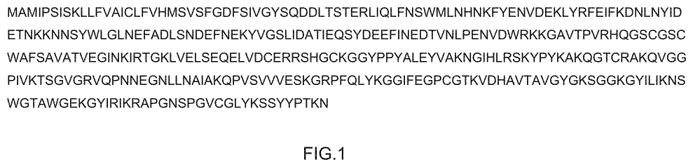

FIG. 2 illustrates the primary amino acid sequence of Carica papaya caricain (GenBank Accession No. X69877).

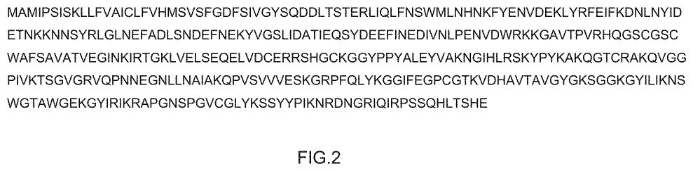

FIG. 3 is a schematic representation of the purification of amylase/trypsin inhibitors from wheat.

FIG. 4 shows the purification of alpha-amylase/trypsin inhibitors from wheat flour. Bands numbered in light grey (8, 9, 10, 11, 12, 13, 14) were confirmed as containing ATI proteins by MS/MS protein sequencing (e.g. see Table 1).

FIG. 5 shows the digestion of wheat flour amylase/trypsin inhibitors (ATI) with a proteolytic preparation comprising Carica papaya endopeptidases (Enzyme 1; ‘Enz 1’), examined by SDS-PAGE. The addition of DTT, pepsin (PEP) or trypsin (Tryp) had no effect as the digestion of ATIs by the proteolytic preparation comprising Carica papaya endopeptidases, which was complete in all cases. Lane 4 “ATI+PEP” refers to ATI+Enz1+PEP and lane 5 “ATI+PEP+DTT” refers to ATI+Enz1+Pep+DTT.

FIG. 6 shows (A) the digestion of wheat flour amylase/trypsin inhibitors (ATI) with a proteolytic preparation comprising Carica papaya endopeptidases (Enzyme 1; ‘Enz 1’), and the resistance of ATIs to digestion using Pepsin or Trypsin, examined by SDS-PAGE, and (B) light scattering analysis of digestion of purified ATIs by the proteolytic preparation comprising Carica papaya endopeptidases.

FIG. 7 shows SDS-PAGE bands from proteolysis of alpha-amylase/trypsin inhibitors at various concentrations using the proteolytic preparation comprising caricain versus a high concentration of pepsin and trypsin.

FIG. 8 shows epitope mapping analysis of ATIs treated with pepsin followed by trypsin in the presence of a preparation comprising Carica papaya endopeptidases, (Enz 1) both at pH 7 or pH 3.

FIG. 9 shows epitope mapping analysis of ATIs treated with pepsin followed by trypsin in the absence of a preparation comprising Carica papaya endopeptidases, (Enz 1) both at pH 7 or pH 3.

FIG. 10 shows epitope mapping analysis of ATIs treated with pepsin followed by trypsin in the presence of a proteolytic preparation comprising Carica papaya endopeptidases (Enz 1) at pH 7.

FIG. 11 shows epitope mapping analysis of ATIs treated with pepsin followed by trypsin in the presence of a proteolytic preparation comprising Carica papaya endopeptidases (Enz 1) at pH 3.

FIG. 12A. Characterization of ATIs digested by trypsin or chymotrypsin and MRM method development for targeted analysis; “Experiment 1”. FIG. 12B shows time course digestion analysis of ATI proteins using a proteolytic preparation comprising caricain in combination with trypsin or chymotrypsin.

FIG. 13 shows the amino acid analysis design to determine cleavage specificity of the proteolytic preparation comprising caricain. P1, P2, P3 and P1′, P2′ and P3′ residues adjacent to the cleavage site were analysed in detail.

FIG. 14 shows the phylogenetic analysis of identified ATI sequences. ATI types and chromosomal locations were determined using the sequence annotations and a sequence alignment against the ATI sequences present in the wheat genome.

FIG. 15 shows epitope mapping analysis in the identified ATI sequences form Baxter and Lancer. Proteins identified in Baxter or Lancer are labelled in the protein ID. Celiac disease associated TLR4 epitopes (Cuccioloni et al., 2017) are highlighted in orange, Bakers asthma-associated epitopes (Walsh 1998) with median affinity levels above 1000 are highlighted in dark green, while epitopes showing median affinity values of 600-800 are highlighted in light blue.

FIG. 16 shows determination of ATI sub-type specific peptide sets using the IDA results of cultivar Baxter. Peptides detected from the identified proteins are presented in the columns. Proteins identified in the discovery phase are shown in the rows. Blue blocks represent peptides that were present in the identified proteins. ATI subtypes and chromosomal positions are used for annotations.

FIG. 17 shows a schematic of changing intensity values for peptide TDLLPHCR in cultivar Baxter.

FIG. 18 shows the overall trends of ATI peptide digestibility in Baxter and Lancer. Peptide peak area values of all peptides included in the analysis are presented in the individual replicates and monitored during the digestion analysis.

FIG. 19 shows changes in the abundance of peptides specific for dimeric ATIs. (A) Representative dimeric ATI sequences identified in Baxter. (B) Quantitative changes in peptides specific for this ATI sub-type. (C) Peptides overlapping with known Baker's asthma epitopes. Overlapping region in the peptide and the epitopes are highlighted in blue.

FIG. 20 shows the monitoring of peptides characteristic on monomeric ATIs. (A) Representative monomeric ATI sequence identified in Baxter. Peptides used in the targeted analysis are labelled with green blocks, Baker's asthma related epitopes are also labelled. (B) Changes in peptide abundance. Peptide regions overlapping with Baker's asthma epitopes are highlighted in blue.

FIG. 21 shows the monitoring of changes of detected CM3-specific peptides. Panel A shows the CM3 ATI sequences detected in both cultivars. Peptides used in the targeted analysis are highlighted as green blocks. TLR4 epitopes are highlighted in yellow. Quantitative changes of peptides underlined in blue in panel A are shown in panel B. Results of peptide quantifications overlapping with the known epitopes (underlined in red in panel A) are shown in panel C. Peptide fragments overlapping with epitopes are highlighted in orange.

FIG. 22 shows P1-P1′ cleavage patterns considering all peptides detected in the sample. (A) Number of P1-P1′ pairs. (B) Frequencies of observed P1-P1′ amino acid characteristics.

FIG. 23 shows the most enriched P1 and P1′ amino acid characteristics detected in the ATI peptides from Filtrate 1. (A) Number of P1-P1′ pairs. (B) Frequencies of observed P1-P1′ amino acid characteristics.

FIG. 24 shows quantitative changes in peptides specific for ATI epitope QECCEQLANIPQQCR.

FIG. 25 shows quantitative changes in peptides specific for ATI epitope IEMPGPPYLAK.

FIG. 26 shows quantitative changes in peptides specific for ATI epitope YFMGPK.

FIG. 27 shows quantitative changes in peptides specific for ATI epitope DCCQQLADINNEWCR.

FIG. 28 shows quantitative changes in peptides specific for ATI epitope VPALPGCRPVLK.

FIG. 29 shows Characterisation of peptides identified from the Baxter flour samples extracted in reducing and non-reducing conditions.

FIG. 30 shows Relative abundance changes of the tryptic ATI peptides digested by a proteolytic preparation comprising Carica papaya endopeptidases (Enz 1/Ccn) in non-reducing conditions. Each experiment was normalized to its own 0 time point values.

FIG. 31 shows Relative changes of the group specific peptides showing effective digestion of ATI groups by a proteolytic preparation comprising Carica papaya endopeptidases (Enz 1/Ccn) both in the presence and absence of pepsin.

FIG. 32 shows Relative abundance changes of the tryptic ATI peptides digested by An-PEP in non-reducing conditions in absence and presence of pepsin. Each experiment was normalized to its own 0 time point values.

FIG. 33 shows Relative changes of the group specific peptides showing An-PEP is not suitable to digest ATIs.

### DETAILED DESCRIPTION

ATIs are albumin proteins found in wheat representing up to 4% of total proteins in grains. They are highly resistant to intestinal proteases and heat and may induce release of pro-inflammatory cytokines from monocytes, macrophages and dendritic cells through activation of a toll-like receptor-4 in Crohn's Disease (CD) and Non-Celiac Gluten Sensitivity (NCGS) patients. ATIs are major allergens in baker's asthma.

ATIs provoke activation of innate immune cells and intestinal inflammation. ATIs activate immunological system through effects on toll-like receptor-4 in CD. It has been demonstrated that mice deficient for TLR4 are protected from intestinal and systemic immune responses during oral intake of ATIs. It is also known that ATIs stimulate monocytes, macrophages and dendritic cells in vitro to produce IL-8, IL-12, TNF, MCP-1 and Regulated on Activation, Normal T-cell Expressed and Secreted (RANTES). ATIs can evoke intestinal inflammation by activating gut and mesenteric lymph node myeloid cells. Accordingly, ATIs can contribute to the activation of innate immune cells in low-level pre-existing small intestinal and colonic inflammation and have a role in the pathophysiology of NCGS. NCGS presents also extra-intestinal symptoms, such as confusion and headache, chronic fatigue, joint/muscle pain, and the exacerbation of pre-existing neurological, psychiatric, or (auto-)immune diseases.

The present inventors have demonstrated that a composition comprising a proteolytic preparation comprising one or more Carica papaya endopeptidases is able to proteolytically cleave immunostimulatory ATIs. For example, Example 2 demonstrates that a proteolytic preparation comprising one or more Carica papaya endopeptidases (Enz 1) completely digests ATIs. Examples 3 and 4 demonstrate that ATIs are resistant to degradation with pepsin and trypsin, and that the proteolytic preparation comprising one or more Carica papaya endopeptidases (Enz 1) completely digested all ATI bands. Example 5 demonstrates that the proteolytic preparation comprising Carica papaya endopeptidases is able to digest B cell and TLR4 epitopes of ATIs. Example 7 demonstrates that the proteolytic preparation comprising Carica papaya endopeptidases is able to cleave monomeric ATIs, dimeric ATIs and CM ATIs.

Accordingly, in one aspect, the present invention provides a method for digesting at least one alpha amylase trypsin inhibitor (ATI), the method comprising contacting an ATI with an effective dose of a composition comprising a proteolytic preparation comprising one or more Carica papaya endopeptidases.

Example 8 demonstrates that a proteolytic preparation comprising one or more Carica papaya endopeptidases as described herein (Enz 1) comprises caricain, multiple isoforms of papain, chymopapain and papaya proteinase 4. The present inventors have also demonstrated that a proteolytic preparation comprising one or more Carica papaya endopeptidases as described herein (Enz 1) also comprises Glutamine cyclotransferase (data not shown).

As used herein the term “digestion” refers to any means, such as proteolysis or proteolytic digestion, of splitting or degrading a protein into smaller peptide fragments, and is used interchangeably herein with the term cleavage. In one embodiment, digestion or degradation ATIs, for example in a gluten-containing foodstuff, results in complete elimination of ATI peptides or immunogenic ATI epitopes, for example following ingestion of an ATI-containing foodstuff, or in a foodstuff. In another embodiment, the “digestion”, or “treating” or “treatment” of an ATI-containing foodstuff results in at least 10% reduction of ATI peptides or immunogenic ATI epitopes, for example following ingestion of an ATI-containing foodstuff, or in a foodstuff. In other embodiments the “digestion”, or “treating” or “treatment” of an ATI-containing foodstuff results in at least a 20, 30, 40, 50, 60, 70, 80, or 90% reduction of ATI peptides or immunogenic ATI epitopes, for example following ingestion of an ATI-containing foodstuff, or in a foodstuff.

As used herein, the term “treating” or “treatment” with respect to a gluten-containing foodstuff means degrading or digesting the foodstuff to reduce the production of toxic gluten oligopeptides when the foodstuff is subsequently ingested and further digestion by a subject. Preferably the subject is a human. In one embodiment, “treating” or “treatment” of a gluten-containing foodstuff results in complete elimination of toxic gluten oligopeptides when the foodstuff is subsequently ingested and further digestion by a subject. In another embodiment, the “treating” or “treatment” of a gluten-containing foodstuff results in at least 10% reduction of toxic gluten oligopeptides when the foodstuff is subsequently ingested and further digestion by a subject. In other embodiments, the “treating” or “treatment” of a gluten-containing foodstuff results in at least 20%, at least 30%, at least 40%, at least 50%, at least 60%, at least 70%, at least 80%, at least 90%, at least 95%, at least 99% or even 100% reduction of toxic gluten oligopeptides when the foodstuff is subsequently ingested and further digestion by a subject.

As used herein the term “alpha amylase trypsin inhibitor” (ATI) is used interchangeably with amylase trypsin inhibitor and includes all structurally and functionally related molecules present in plants that act as triggers of innate immune activation, for example, having TLR4-stimulating activities in foodstuffs. The term includes monomeric and (non-covalently linked) dimeric and tetrameric forms. Exemplary ATIs include the 12 kDa monomeric inhibitors, also known as 0.28 proteins, the 24 kDa homodimeric inhibitors, also known as the 0.19 and 0.53 proteins, and the 60 kDa heterotetrameric inhibitors. This 60 kDa group includes proteins characterized by specific solubility in chloroform/methanol (CM) mixtures, that accounts for their belonging to the so-called CM protein types. Exemplary CM ATIs include CM1, CM2, CM3, CM16 and CM17.

As used herein the term epitope includes epitopes of ATIs, including B cell epitopes and TLR4 epitopes. Exemplary B cell epitopes are recited in Table 2, and include

AASVPE
ADINNE
ALTGCR
AMVKLQ
AVLRDC
AYPDV
CQQLAD
CRAMVK
CVGSQV
CYGDWA
DCCQQL
ELGVRE
EVMKLT
GCRKEV
GDRAGV
GDWAAY
GKEVLP
GSQVPE
GVCYGD
GVREGK
INNEWC
KVPIPN
LPGCRK
LQCVGS
LRDCCQ
LRSVYQ
LSSMLR
LTAASV
MKLTAA
NEWCRC
PATGYK
PEAVLR
PEVCKV
PSGDRA
QLADIN
QVPEAV
RAGVCY
RCGDLS
REGKEV
RKEVMK
SGPWSW
SMLRSV
SVPEVC
SVYQEL
TGCRAM
TGYKVS
VCKVPI
VKLQCV
VSALTG
WAAYPD
WCRCGD
YKVSAL
YQELGV
PWSWCD
SWCDPA
SWCDPATGYKVSALTGCRAMV
DCCQQLAHISEWCR
EHGAQEGQAGTGAFPR
CGALYSMLDSMYK
LQCNGSQVPEAVLR
LPIVVDASGDGAYVCK
LTAASITAVCR
SGPWMCYPGQAFQVPALPACRPLLR
DCCQQLADISEWCR
EHGVSEGQAGTGAFPSCR
SGPWMCYPGQAFQVPALPGCRPLLK
ECCQQLADISEWCR
LTAASITAVCK
SGPWMCYPGYAFK
VPALPGCRPVLK
IETPGSPYLAK
SDPNSSVLK
ELYDASQHCR
EYVAQQTCGVGIVGSPVSTEPGN TPR
TSDPNSGVLK
VLVTPGHCNVMTVHNTPYCLGLD I
IEMPGPPYLAK
NYVEEQACR
QECCEQLANIPQQCR
SRPDQSGLMELPGCPR
YFMGPK
EVQMDFVR;
SVYQEIGVR;
GPSLPMLVK
SDPNSSVLK;
EHGAQEGQAGTGAFPR
EFIAGIVGR

Exemplary TLR4 epitopes of ATIs include LPEWMTSAS and SGNVGESGLI.

In one embodiment the epitope is comprises one or more cysteine amino acids.

It will be appreciated that this list of ATIs is not intended to be exhaustive, furthermore, oligomers, biologically active fragments, variants and analogues thereof are also intended to be included as part of these examples.

In some embodiments of the invention ATIs are derived from cereals such as wheat, wheat germ, rye, barley, bulgur, cuscous, farina, graham flour, kamut matzo, semolina, spelt, or triticale.

As used herein the term “contacting” refers to the bringing together of indicated moieties (e.g. an ATI or an ATI epitope described herein and a proteolytic preparation comprising one or more Carica papaya endopeptidases) in vitro or in vivo.

The present inventors have demonstrated in the Examples that a proteolytic preparation comprising one or more Carica papaya endopeptidases as described herein (Enz 1) is able to digest ATIs at physiologically relevant pH (e.g. the pH of the stomach and/or small intestine), and in the presence of pepsin and trypsin. The present inventors have also demonstrated that the proteolytic preparation comprising one or more Carica papaya endopeptidases as described herein (Enz 1) is enzymatically active following administration to humans (data not shown).

A composition comprising a proteolytic preparation comprising one or more Carica papaya endopeptidases as described herein is typically administered in an effective amount. By the term “effective amount” (for example a “therapeutically effective amount” or a “pharmaceutically effective amount”) as used herein refers to an amount of a composition comprising a proteolytic preparation comprising one or more Carica papaya endopeptidases that allows an effective digestion of at least one ATI or an effective response to treatment. Said “effective amount” will vary from subject to subject, depending on the age and general condition of the individual and with the factors such as the particular condition being treated or prevented, the duration of the treatment, previous treatments and the nature and pre-existing duration of the condition.

As used herein the term “composition” includes pharmaceutical compositions and dietary supplements and the like. The term “pharmaceutical composition” refers to a medicament or a drug, while the term “dietary supplement” refers to a small amount of an active principle for supplementation of a human diet packaged in single or multidose units. The dietary supplement or pharmaceutical composition according to the present invention further comprises pharmaceutically acceptable excipients. “Excipients” mean excipients, carriers, or diluents, including, but not limited to, water, gelatin of any origin, vegetable gums, ligninsulfonate, talc, sugars, starch, cellulose, microcrystalline cellulose, gum arabic, vegetable oils, polyalkylene glycols, flavouring agents, preservatives, stabilizers, emulsifying agents, buffers, lubricants, colorants, wetting agents, filters, and the like. The carrier material can be organic or inorganic inert carrier material suitable for oral/parenteral/injectable administration. The dietary supplement or pharmaceutical composition according to the present invention may be in any galenic form that is suitable for administering to humans, but solid or liquid oral forms are preferred, e.g. in solid form, such as additives/supplements for food, tablets, pills, granules, dragees, capsules, gummy formulations, and effervescent formulations such as powders and tablets. The dietary and pharmaceutical compositions may be in the form of controlled (decayed) release formulations

In all the embodiments of the present invention, the dietary supplement or pharmaceutical composition according to the present invention is preferably in the form of a tablet, a capsule, a sachet, or any other dosage form including liquid formulation. More preferably, it is in the form of a tablet or a capsule. The capsules, tablets or sachets or other dosage forms may be in a container which may take any conventional form. For example, the dosage forms may be sold in a jar, bottle, tin box, pot, dispenser, sachet or the like which contains the dosage forms in a predetermined quantity, such as a 30-day supply, a 60-day supply, a 90-day supply or in whatever quantity which is desired. Additionally, and optionally, the capsules may be in a blister pack, wherein each blister contains a predetermined number of capsules, usually a single dose (typically 1-4 capsules). The arrangement of the number of capsules in a blister, the number of blisters on a single blister pack strip, and the number of blister pack strips which are sold in a group may be any convenient amounts or configurations.

The dietary or pharmaceutical compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, filters, co-compounds, dispersing agents, wetting agents, processing aids (solvents), flowing agents, taste masking agents, weighting agents, gelling agents, gel forming agents, antioxidants and antimicrobials.

In the context of the present invention, a pharmaceutical composition is sold with or without a prescription, while a dietary supplement is to be sold over the counter without medical prescription and is to be considered as food.

The compositions according to the present invention, as hereinbefore described, may be in the form of a pharmaceutical composition, in which the composition further includes a pharmaceutically acceptable carrier, excipient, diluent and/or adjuvant. Pharmaceutical compositions of the present invention may be employed alone or in conjunction with other compounds, such as therapeutic compounds.

As used herein, the phrase “pharmaceutically acceptable carrier” includes, but is not limited to, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Typically, the route of administration is parenteral, including oral (e.g., ingestion, inhalation) or rectal. Solutions or suspensions used for parenteral application can include one or more of the following components: a sterile diluent such as water for injection, saline solution, fixed oils, polyethylene glycols, glycerine, propylene glycol or other synthetic solvents; antibacterial agents such as methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Generally, the pharmaceutical composition is stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, or liquid polyetheylene glycol, and the like), and suitable mixtures thereof. The proper fluidity can be maintained, for example, by the use of a coating such as lecithin, by the maintenance of the required particle size in the case of a dispersion or by the use of surfactants. Prevention of the action of microorganisms can be achieved by incorporation of various antibacterial and antifungal agents, for example, parabens, chlorobutanol, phenol, ascorbic acid, thimerosal, and the like. In many cases, it will be preferable to include isotonic agents, for example, sugar, sodium chloride or polyalcohols such as mannitol, or sorbitol, in the composition.

Oral compositions generally comprise an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or modified corn starch; a lubricant such as magnesium stearate or other stearates; a glidant such as colloidal silicon dioxide; a sweetening agent such as sucrose or saccharin; or a flavouring agent such as peppermint, methyl salicylate, or orange flavouring.

In one embodiment, the compositions of the present invention are prepared with carriers that will protect the compositions according to the present invention against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers.

It is advantageous to formulate oral or parenteral compositions in dosage unit form for ease of administration and uniformity of dosage. “Dosage unit form”, as used herein, refers to physically discrete units suited as unitary dosages for the subject to be treated; each unit containing a predetermined quantity of active compound calculated to produce the desired therapeutic effect in association with the required pharmaceutical carrier. The toxicity and therapeutic efficacy of such compounds can be determined by standard pharmaceutical procedures including in vitro assays, cell cultures or experimental animals, e.g., for determining the LD50 (the dose lethal to 50% of the population) and the ED50 (the dose therapeutically effective in 50% of the population) depending on the compound studied. The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the in vitro studies, cell culture assays and animal studies can be used in formulating a range of dosages for use in humans. The dosage lies preferably within a range of circulating concentrations that include the ED50 with little or no toxicity. The dosage may vary within this range depending upon the dosage form employed and the route of administration utilized. The therapeutically effective dose of an enzyme can be estimated initially from in vitro assays. Such information can be used to more accurately determine useful doses in humans.

Those of skill in the art will readily appreciate that dose levels can vary as a function of the specific enzyme, the severity of the symptoms and the susceptibility of the subject to side effects. In some embodiments, dosages for a given enzyme are readily determinable by those of skill in the art by a variety of means. An exemplary means is to measure the biological activity of a given compound required to overcome the symptoms.

The skilled artisan will appreciate that certain factors may influence the dosage and timing required to effectively treat a subject, including the activity of the specific compound employed, the age, body weight, general health, gender, and diet of the subject, the time of administration, the route of administration, the rate of excretion, any drug combination, the degree of expression or activity to be modulated, sensitivity to gluten, previous treatments and other diseases present.

The pharmaceutical compositions can be included in a container, pack, or dispenser together with instructions for administration.

For oral preparations, the compositions according to the present invention can be used alone or in combination with appropriate additives to make tablets, powders, granules or capsules, for example, with conventional additives, such as lactose, mannitol, corn starch or potato starch; with binders, such as crystalline cellulose, cellulose variants, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavouring agents.

In one embodiment of the present invention, the oral formulations comprise enteric coatings, so that the active agent is delivered to the intestinal tract. Enteric formulations are often used to protect an active ingredient from the strongly acid contents of the stomach. Such formulations can be created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate, hydroxypropyl methylcellulose acetate succinate, methacrylate copolymers, cellulose acetate phthalate and acrylic coating systems such as Acryl-Ease (Colorcon).

Other enteric formulations comprise engineered polymer microspheres made of biologically erodible polymers, which display strong adhesive interactions with gastrointestinal mucus and cellular linings and can traverse both the mucosal absorptive epithelium and the follicle-associated epithelium covering the lymphoid tissue of Peyer's patches. The polymers maintain contact with intestinal epithelium for extended periods of time and actually penetrate it, through and between cells (see, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71 (3):307-18).

As used herein, the term “proteolytic preparation comprising one or more Carica papaya endopeptidases” is used to refer to an enzyme preparation that contains papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5). An exemplary composition comprising a proteolytic preparation comprising one or more Carica papaya endopeptidases is Enz 1 as described herein.

Example 8 demonstrates that a proteolytic preparation comprising one or more Carica papaya endopeptidases as described herein (Enz 1) comprises caricain, multiple isoforms of papain, chymopapain and papaya proteinase 4. The present inventors have also demonstrated that a proteolytic preparation comprising one or more Carica papaya endopeptidases as described herein (Enz 1) also comprises Glutamine cyclotransferase (data not shown).

Accordingly, in one embodiment, the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5).

A proteolytic preparation comprising one or more Carica papaya endopeptidases can be tested for its ability to degrade ATIs by any one or more of the procedures herein described. It will be understood by the skilled addressee that methods of preparing proteolytic preparation comprising one or more Carica papaya endopeptidases may include isolating and purifying Carica papaya endopeptidases from papaya latex, or from any other suitable source, and will be such that at least some enzyme activity of the isolated peptidase is retained so as to provide for the methods of degrading α-amylase/trypsin inhibitors, and to the treatment and/or prophylaxis of a α-amylase/trypsin inhibitor (ATI) mediated conditions. One skilled in the art would appreciate that there are numerous methods and techniques for measuring qualitatively and/or quantitatively the ability of the at least partially purified caricain to degrade ATI-peptides, either in vitro or in vivo, as herein described.

As used herein and with reference to of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5), the term “biologically active fragment” typically refers to a fragment that retains its ability to detoxify gluten peptides, in vitro or in vivo.

Peptidase fragments of interest include, but are not limited to, fragments of at least about 20 contiguous amino acids, more usually at least about 50 contiguous amino acids, and may comprise 100 or more amino acids, up to the complete protein, and may extend further to include additional sequences. In each case, the key criterion is whether the fragment retains the ability to modify the toxic oligopeptides that contribute to a condition arising from gluten intolerance.

As used herein, the term “native” preferably refers to papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5) having an amino acid sequence that occurs in nature (e.g., a natural protein). Such sequences may generally be identified using techniques well known to those skilled in the art in identifying peptidase activity.

As used herein and with reference to papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5), the term “analogue” typically denotes a peptidase that has an amino acid sequence that is substantially identical to the amino acid sequence of naturally occurring papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5).

The term “substantially identical”, as used herein with reference to an analogue, typically denotes a substitution or addition of one or more amino acids such that the resulting analogue has at least some of the biological activity of the naturally occurring enzyme. Analogues may be naturally occurring, such as an allelic variant or an mRNA splice variant, or they may be constructed using synthetic or recombinant techniques available to one skilled in the art.

As used herein and with reference to papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5), the term “variant” typically denotes an enzyme that exhibits an amino acid sequence that is at least 80% identical to the native enzyme. Also contemplated are embodiments in which a variant comprises an amino acid sequence that is at least 90% identical, optionally at least 95% identical, optionally at least 98% identical, optionally at least 99% identical, or optionally at least 99.9% identical to the native molecule. Percent identity may be determined by visual inspection and/or mathematical calculation by methods known to those skilled in the art. Variants may be naturally occurring, synthetic or recombinant.

In one embodiment of the present invention, a variant of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5) includes an enzyme that is substantially homologous to the native form of the enzyme, but which has an amino acid sequence different from that of the native form because of one or more deletions, insertions or substitutions. Certain embodiments include amino acids that comprise from one to ten deletions, insertions or substitutions of amino acid residues when compared to a native sequence. A given sequence may be replaced, for example, by a residue having similar physiochemical characteristics. Examples of such conservative substitution of one aliphatic residue for another, such as Ile, Val, Leu or Ala for one another; substitution of one polar residue for another, such as between Lys and Arg, or Glu and Asp, or Gln and Asn; or substitutions of one aromatic residue for another, such as Phe, Trp or Tyr for one another. Other conservative substitutions, e.g., involving substitutions of entire regions having similar hydrophobicity characteristics, are well known in the art. Variants may also be generated by the truncation of a native peptidase amino acid. Further variants encompassed by the present invention include, but are not limited to, deglycosylated amino acids, or fragments thereof, or those amino acids demonstrating increased glycosylation when compared to the native enzyme.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). Thus, an amino acid residue of caricain is preferably replaced with another amino acid residue from the same side chain family. In a preferred embodiment, mutations can be introduced randomly along all or part of the enzyme coding sequence, such as by saturation mutagenesis. The resultant mutants can be screened to identify variants that demonstrate at least some of the biological activity of the native enzyme. Following mutagenesis, the encoded protein can be expressed recombinantly and the activity of the enzyme can be determined by the methods described herein.

Also envisaged are modifications that do not alter the primary sequence of the native form of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5), including, but not limited to, chemical derivatization of proteins (e.g., acetylation or carboxylation), glycosylation (e.g., those made by modifying the glycosylation patterns of a protein during its synthesis and processing or in further processing steps), as well as sequences that have phosphorylated amino acid residues (e.g., phosphotyrosine, phosphoserine, or phosphothreonine).

Also useful in the practice of the present invention is papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5) that has been modified using molecular biological techniques and/or chemistry so as to improve their resistance to proteolytic degradation and/or to acidic conditions such as those found in the stomach, and to optimize solubility properties or to render them more suitable as a therapeutic agent. Analogs of such proteins include those containing residues other than naturally occurring L-amino acids (e.g., D-amino acids or non-naturally occurring synthetic amino acids).

The papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5) enzymes according to the present invention may be prepared by in vitro synthesis using conventional methods as known in the art. Various commercial synthetic apparatuses are available, for example, automated synthesizers (e.g., CS936X Peptide Synthesizer, CSBio Company, Inc.). Using such synthesizers, a skilled person can readily substitute for the naturally occurring amino acids one or more unnatural amino acids. The particular sequence and the manner of preparation will be determined by convenience, economics, purity required, and the like. If desired, various groups can be introduced into the protein during synthesis that allow for linking to other molecules or to a surface. For example, cysteines can be used to make thioethers, histidines can be used for linking to a metal ion complex, carboxyl groups can be used for forming amides or esters, amino groups can be used for forming amides, and the like.

As used herein, the term “functional equivalent thereof” refers to a sequence that has an analogous function to the sequence of which it is a functional equivalent. By “analogous function” is meant that the sequences share a common function, for example, in encoding papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5), or a biologically active fragment, analogue or variant thereof. In some embodiments, a functionally equivalent sequence may exhibit sequence identity with the sequence of which it is a functional equivalent. The sequence identity between the functional equivalent and the sequence of which it is a functional equivalent may be at least 50% across the length of the functional equivalent, at least 60% across the length of the functional equivalent or greater than 70%, 80%, 85%, 90%, 95%, 96%, 97%, 98% or 99% across the length of the functional equivalent.

In one embodiment, the proteolytic preparation comprising one or more Carica papaya endopeptidases as described herein comprises about 5% w/w to about 95% w/w caricain (EC 3.4.22.30), or a biologically active fragment, analogue or variant thereof, of total weight of the proteolytic preparation.

In one embodiment the present invention provides a composition as described herein, wherein the composition comprises a proteolytic preparation comprising one or more Carica papaya endopeptidases as described herein, wherein the proteolytic preparation comprising one or more Carica papaya endopeptidases comprises about 5% w/w to about 95% w/w caricain (EC 3.4.22.30), or a biologically active fragment, analogue or variant thereof, of total weight of the proteolytic preparation.

In one embodiment, the proteolytic preparation comprising one or more Carica papaya endopeptidases as described herein comprises at least 5, 6, 7, 9, 10, 11, 12, 13, 14, 15, 16, 17, 17.3, 18, 19, 20, 21, 22, 23, 24, 25, 30, 35, 40, 45, 50, 55, 60, 65, 70, 75, 80, 85, 90, 95% m/m caricain (EC 3.4.22.30), or a biologically active fragment, analogue or variant thereof, of the proteolytic preparation.

In one embodiment a proteolytic preparation comprising one or more Carica papaya endopeptidases as described herein is prepared by filtering either papaya latex or solubilised dried papaya latex in water, to result in a filtrate which is concentrated, and which is sterile filtered (e.g. filtered using a 0.45 u filtration cartridge) to form a concentrate which is stray dried, sieved (e.g. using a 200-350 u stainless steel sieve) and filled and packaged.

Notably, the disease causing potency of ATIs ingested with wheat, barley, or rye (gluten-containing cereals), as well as with some non-gluten containing staples, is not limited to the gastrointestinal tract, but is also likely implicated to affect other extraintestinal diseases (Catassi et al., Nutrients 7:4966-4977, 2015; Fasano et al., Gastroenterology 148:1 195-1204, 2015; Schuppan et al., Best Practice & Research: Clinical Gastroenterology 29:469-76, 2015). Broad evidence indicates that nutritional ATIs induce low grade but significant inflammation in the small intestine, colon, and. the surrounding mesenteric lymph nodes. Notably, nutritional ATIs exacerbate inflammatory diseases in general, as illustrated in mouse models of inflammatory bowel disease, of multiple sclerosis, of systemic lupus erythematosus, of non-alcoholic steatohepatitis, and of allergic asthma.

It has been demonstrated that ATIs are highly heat resistant and are present in foodstuffs and heat-treated liquids. Accordingly, in another embodiment, the present invention provides a method for digesting at least one alpha amylase trypsin inhibitor (ATI) in an ATI-containing foodstuff, the method comprises contacting the ATI-containing foodstuff with an effective dose of a composition comprising a proteolytic preparation comprising one or more Carica papaya endopeptidases.

In one embodiment, provided herein is a foodstuff comprising a composition comprising a proteolytic preparation comprising one or more Carica papaya endopeptidases as described herein.

In one embodiment, the foodstuff is a gluten free or gluten reduced foodstuff.

In one embodiment, provided herein is a method for degrading an ATI in a gluten-free ATI-containing foodstuff, the method comprises contacting the gluten-free ATI-containing foodstuff with an effective dose of a composition comprising a proteolytic preparation comprising one or more Carica papaya endopeptidases as described herein.

In another embodiment, provided herein is a method for degrading an ATI in an ATI-containing foodstuff with an effective dose of a composition comprising a proteolytic preparation comprising one or more Carica papaya endopeptidases as described herein.

In one embodiment, provided herein is a method for detoxifying an ATI, the method comprising contacting the ATI-containing foodstuff with an effective amount a composition comprising a proteolytic preparation comprising one or more Carica papaya endopeptidases as described herein.

In one embodiment, provided herein is a gluten-free foodstuff composition comprising a gluten-containing foodstuff in an admixture an effective amount a composition comprising a proteolytic preparation comprising one or more Carica papaya endopeptidases as described herein.

In one embodiment of the degrading method described, the contacting of the foodstuff with a composition comprising a proteolytic preparation comprising one or more Carica papaya endopeptidases as described herein is performed in vitro prior to consumption of the foodstuff (e.g. ATI containing foodstuff).

In one embodiment of the degrading method described, the contacting of the foodstuff with a composition comprising a proteolytic preparation comprising one or more Carica papaya endopeptidases as described herein is performed in vivo concurrent with or after consumption of the foodstuff (e.g. ATI containing foodstuff).

In one embodiment of the treatment method described, the administering of a composition comprising a proteolytic preparation comprising one or more Carica papaya endopeptidases as described herein is performed in vivo concurrent with or after consumption of the ATI-containing food stuff or the ATI-containing foodstuff respectively.

In one embodiment of the treatment method described, the administering of a composition comprising a proteolytic preparation comprising one or more Carica papaya endopeptidases as described herein is performed in vitro prior to and also concurrent with or after consumption of the ATI-containing foodstuff.

In one embodiment the present invention provides a method as described herein, wherein the ATI is contacted with the proteolytic preparation comprising one or more Carica papaya endopeptidases under conditions sufficient to cause digestion of the ATI by hydrolysis.

In one embodiment, the conditions are in vivo conditions following administration of the proteolytic preparation comprising one or more Carica papaya endopeptidases under conditions sufficient to cause digestion of an ATI in a subject, for example, prior to, with, or following, eating of an ATI-containing foodstuff.

In a preferred embodiment the the proteolytic preparation comprising one or more Carica papaya endopeptidases is administered orally within 1 hour prior to or following ingestion of a meal.

In one embodiment of the methods described herein, the at least one ATI is an ATI derived from a wheat, barley, rye or oat species.

In one embodiment of the methods described herein, the at least one ATI is an ATI derived from a spelt, khorasan, emmer, einkorn, and triticale species.

In a preferred embodiment of the methods described herein, the at least one ATI is a wheat ATI.

In one embodiment of the methods described herein, at least one epitope of the at least one ATI is digested.

In one embodiment, the at least one epitope is selected from;
a) an Alpha-amylase inhibitor precursor (CIII) (WMAI-1) B cell epitope selected from the group consisting of:

AASVPE
ADINNE
ALTGCR
AMVKLQ
AVLRDC
AYPDV
CQQLAD
CRAMVK
CVGSQV
CYGDWA
DCCQQL
ELGVRE
EVMKLT
GCRKEV
GDRAGV
GDWAAY
GKEVLP
GSQVPE
GVCYGD
GVREGK
INNEWC
KVPIPN
LPGCRK
LQCVGS
LRDCCQ
LRSVYQ
LSSMLR
LTAASV
MKLTAA
NEWCRC
PATGYK
PEAVLR
PEVCKV
PSGDRA
QLADIN
QVPEAV
RAGVCY
RCGDLS
REGKEV
RKEVMK
SGPWSW
SMLRSV
SVPEVC
SVYQEL
TGCRAM
TGYKVS
VCKVPI
VKLQCV
VSALTG
WAAYPD
WCRCGD
YKVSAL
YQELGV;

b) a putative alpha-amylase inhibitor B cell epitope selected from the group consisting of PWSWCD, SWCDPA, and SWCDPATGYKVSALTGCRAMV;
c) a peptide comprising an ATI epitope, said peptide selected from the group consisting of:

DCCQQLAHISEWCR
EHGAQEGQAGTGAFPR
CGALYSMLDSMYK
LQCNGSQVPEAVLR
LPIVVDASGDGAYVCK
LTAASITAVCR
SGPWMCYPGQAFQVPALPACRPL LR
DCCQQLADISEWCR
EHGVSEGQAGTGAFPSCR
SGPWMCYPGQAFQVPALPGCRPL LK
ECCQQLADISEWCR
LTAASITAVCK
SGPWMCYPGYAFK
VPALPGCRPVLK;

d) a CM16 and/or CM17 ATI epitope selected from the group consisting of:

IETPGSPYLAK
SDPNSSVLK
ELYDASQHCR
EYVAQQTCGVGIVGSPVSTEPGN TPR
TSDPNSGVLK
VLVTPGHCNVMTVHNTPYCLGLD I
IEMPGPPYLAK
NYVEEQACR
QECCEQLANIPQQCR
SRPDQSGLMELPGCPR
YFMGPK
EVQMDFVR;

e) a monomeric ATI epitope selected from the group consisting of:

EVLPGCR
CGDLSSMLR
DCCQQLADINNEWCR
VSALTGCR
SVYQELGVR
SHNSGPWSWCDPATGYK
LTAASVPEVCK
LQCVGSQVPEAVLR
VPIPNPSGDR
SVYQEIGVR;

f) a CM3 ATI epitope selected from the group consisting of:

TDLLPHCR;
LPEWMTSASIYSPGKPYLAK
EMQWDFVR
LYCCQELAEISQQCR
DLPGCPR
LLVAPGQCNLATIHNVR
DYVLQQTCGTFTPGSK
YFIALPVPSQPVDPR
SGNVGESGLIDLPGCPR
LPEWMTSASIFSPMKPYLAK
LYCCQELAEIPQQCR
QMQWDFVR

and
g) a CM Hagerman or CM1 ATI epitope selected from the group consisting of:

GPSLPMLVK
SDPNSSVLK;

and
h) a dimeric ATI epitope selected from the group consisting of:

EHGAQEGQAGTGAFPR
EFIAGIVGR.

In one embodiment, the present invention provides a method described herein, wherein the proteolytic preparation is derived from Carica papaya oleoresin.

In one embodiment of the present invention, a proteolytic preparation comprising one or more Carica papaya endopeptidases as described herein is at least partially purified from a natural source (e.g., papaya latex).

Papaya, the fruit of the tree Carica papaya, in the genus Carica, is also known as mamāo, tree melon, fruta bomba, lechosa or pawpaw. Methods useful for the isolation of a proteolytic preparation comprising one or more Carica papaya endopeptidases as described herein from a natural source such as papaya latex include, but are not limited to, solid-liquid extraction, liquid-liquid extraction, solid-phase extraction, membrane filtration, ultrafiltration, dialysis, electrophoresis, solvent concentration, centrifugation, ultracentrifugation, liquid or gas phase chromatography (including size exclusion chromatography, affinity chromatography, etc) with or without high pressure, lyophilisation, evaporation, precipitation with various “carriers” (e.g., antibodies), crystallization, and any combination thereof. The skilled addressee would understand how to use such options, in a sequential fashion, in order to enrich each successive fraction for the Carica papaya endopeptidases as described herein by following its activity throughout the purification procedure. The activity of the proteolytic preparation comprising one or more Carica papaya endopeptidases as described herein can be measured using a variety of methods including those as described herein in relation to digestion of ATIs

Solid-liquid extraction includes, but is not limited to, the use of various solvents, vortex shakers, ultrasounds and other means to enhance extraction, as well as recovery by filtration, centrifugation and related methods as described in the art (see, e.g., Cannell R J P, Natural Products Isolation, Humana Press, 1998). Examples of solvents that may be used include, but are not limited to, hydrocarbon solvents, chlorinated solvents, organic esters, organic ethers, alcohols, water, and combinations thereof.

Liquid-liquid extraction includes, but is not limited to, the use of solvents known in the art such as hydrocarbon solvents, chlorinated solvents, organic esters, organic ethers, alcohols, water, various aqueous solutions, and combinations thereof. The liquid-liquid extraction can be facilitated manually, or it can be automated (completely or in part), and the solvent can be removed and/or concentrated by standard techniques in the art.

Membrane, reverse osmosis and ultrafiltration include, but are not limited to, the use of various types of membranes known in the art, as well as the use of pressure, vacuum, centrifugal force, and/or other means that can be utilised in membrane and ultrafiltration processes.

Dialysis typically includes the use of membranes having a molecular weight cut-off that is selective for the removal of various constituents from the natural source so as to increase the relative purity of one or more Carica papaya endopeptidases as described herein in a sample. The present invention also encompassed the recovery of purified and/or fractionated extracts from either the dialysate or the retentate by various means known in the art including, but not limited to, lyophilization and crystallization.

Chromatography includes, but is not limited to, the use of regular column chromatography, flash chromatography, high performance liquid chromatography (HPLC), medium pressure liquid chromatography (MPLC), supercritical fluid chromatography (SFC), countercurrent chromatography (CCC), moving bed chromatography, simulated moving bed chromatography, expanded bed chromatography, and planar chromatography. Examples of sorbents that may be used in chromatography include, but are not limited to, silica gel, alumina, fluorisil, cellulose and modified cellulose, various modified silica gels, ion-exchange resins, size exclusion gels, chemically modified gels, and other sorbents known to those skilled in the art. The present invention also includes the use of two or more salt gradients to effect the fractionation and/or partial purification of one or more Carica papaya endopeptidases as described herein by chromatographic methods. When water or an aqueous phase is used, it may contain varying amounts of inorganic or organic salts, and/or the pH may be adjusted to different values with an acid or a base such that fractionation and/or purification is enhanced.

The process of at least partially purifying one or more Carica papaya endopeptidases as described herein from a natural source may also include the concentration one or more Carica papaya endopeptidases as described herein by solvent removal of the original extract and/or fractionated extract, and/or purified extract. The techniques of solvent removal are known to those skilled in the art and include, but are not limited to, rotary evaporation, distillation (normal and reduced pressure), centrifugal vacuum evaporation (speed-vac), lyophilization and combinations thereof.

When referring to one or more Carica papaya endopeptidases of the invention, the term “at least partially purified” typically means a composition which is partially to completely free of other components (e.g., other proteins, nucleic acids, lipids, carbohydrates) with which the peptides, proteins or analogs are associated in a non-purified, e.g., native state or environment. The at least partially purified peptides and proteins can generally be in a homogeneous or nearly homogenous state, although it can be either in a dry state or in an aqueous solution. Purity and homogeneity are typically determined using analytical chemistry techniques such as polyacrylamide gel electrophoresis or high-performance liquid chromatography. In one embodiment, peptides such as the one or more Carica papaya endopeptidases can be further purified using routine and well-known methods, such as those described herein.

The present invention provides example assays for determining ATI activity of a composition or a proteolytic preparation comprising one or more Carica papaya endopeptidases as described herein

In one embodiment, the present invention provides a method described herein, wherein the proteolytic preparation is derived from Carica papaya latex.

In one embodiment, the present invention provides a method described herein, wherein the composition further comprises one or more excipients.

In one embodiment, the present invention provides a method described herein, wherein the method is performed in vitro or in vivo.

In another embodiment, the present invention also relates to a dietary supplement or a pharmaceutical composition comprising a proteolytic preparation comprising one or more Carica papaya endopeptidases as described herein for use as a medicament. Thus the present invention relates to the use of a dietary supplement or a pharmaceutical composition comprising a proteolytic preparation comprising one or more Carica papaya endopeptidases as described herein as a medicament for the treatment of diseases. Preferably, the medicament is for the treatment of a patient suffering from an ATI-mediated disease or condition.

In one embodiment, the present invention provides a method described herein, wherein the composition is administered to a human.

ATI peptides and immunogenic ATI epitopes are causative of a number of human diseases, such as nonceliac wheat sensitivity (NCWS), Baker's asthma, autoimmune diseases and metabolic disorders. Accordingly, the ability to reduce ATI peptides and immunogenic ATI epitopes, or completely digest ATI peptides and immunogenic ATI epitopes, will prevent ATI peptides and immunogenic ATI epitopes interacting with the immune system of a subject administered an effective amount of a composition comprising a proteolytic preparation comprising one or more Carica papaya endopeptidases. For example, the present inventors have demonstrated that a composition comprising a proteolytic preparation comprising one or more Carica papaya endopeptidases is able to proteolytically cleave immunostimulatory ATIs. For example, Example 2 demonstrates that a proteolytic preparation comprising one or more Carica papaya endopeptidases (Enz 1) completely digests ATIs. Examples 3 and 4 demonstrate that ATIs are resistant to degradation with pepsin and trypsin, and that the proteolytic preparation comprising one or more Carica papaya endopeptidases (Enz 1) completely digested all ATI bands. Example 5 demonstrates that the proteolytic preparation comprising Carica papaya endopeptidases is able to digest B cell and TLR4 epitopes of ATIs. Example 7 demonstrates that the proteolytic preparation comprising Carica papaya endopeptidases is able to cleave monomeric ATIs, dimeric ATIs and CM ATIs.

Accordingly, in one embodiment the present invention provides a method for the treatment and/or prevention of an alpha-amylase/trypsin inhibitor (ATI) mediated condition, comprising administering to a subject in need thereof an effective amount of a composition comprising a proteolytic preparation comprising one or more Carica papaya endopeptidases.

ATIs are responsible for manifestations of mainly extra-intestinal symptoms of non-celiac non-allergy wheat sensitivity. ATI are major allergens in baker's asthma, a classical IgE mediated allergy. In the gut ATI are able to stimulate immune cells residing in the lamina propria and mesenteric lymph nodes through TLR4 binding and stimulation, and the emigration of the activated myeloid cells. The innate immune receptor TLR4 recognizes damage and pathogen associated patterns (DAMPs/PAMPs), like lipopolysaccharides (LPS), which are major components in the outer membrane of gram-negative bacteria. Upon stimulation, the receptor triggers an NF-κB dependent cascade leading to the release of pro-inflammatory cytokines. Importantly, ATI can trigger TLR4 by direct interaction, and provoke innate immunity. In a mouse model of inflammatory bowel disease, ATI enhanced the dextran sodium sulfate-induced intestinal inflammation by increasing the number of activated macrophages and dendritic cells in all sections of the intestine, the lamina propria, and especially in the mesenteric lymph nodes. It has also been demonstrated in mouse studies on experimental airway inflammation that ATI-enriched diets not only enhanced allergen-induced intestinal, but also lung allergic responses in an IgE- and TLR4-dependent manner. Thus, the adjuvant effect of ATI is not limited to the intestine, but can also be observed for other organs, fueling ongoing inflammation.

In one embodiment, the condition is a wheat alpha-amylase/trypsin inhibitor (ATI) mediated condition.

In another embodiment, the alpha-amylase/trypsin inhibitor (ATI) mediated condition is wheat allergy, non-celiac gluten/wheat sensitivity, irritable bowel syndrome or baker's asthma.

In another embodiment the present invention provides a method as described herein wherein the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5). In one embodiment, the proteolytic preparation comprises is derived from Carica papaya oleoresin. In another embodiment, the proteolytic preparation comprises is derived from Carica papaya latex. In another embodiment, the composition further comprises one or more pharmaceutically acceptable excipients. In one embodiment the composition is administered orally. In another embodiment the composition is enterically coated. In a preferred embodiment, the subject is a human subject.

In another aspect, the present invention provides a use of a composition comprising a proteolytic preparation comprising one or more Carica papaya endopeptidases in the manufacture of a medicament for the treatment and/or prophylaxis of an α-amylase/trypsin inhibitor (ATI) mediated condition. In one embodiment, the condition is a wheat alpha-amylase/trypsin inhibitor (ATI) mediated condition.

In another embodiment, the alpha-amylase/trypsin inhibitor (ATI) mediated condition is wheat allergy, non-celiac gluten/wheat sensitivity, irritable bowel syndrome or baker's asthma.

In one embodiment, the present invention provides a use as described herein, wherein the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5). In one embodiment, the proteolytic preparation comprises is derived from Carica papaya oleoresin. In another embodiment, the proteolytic preparation comprises is derived from Carica papaya latex. In another embodiment, the composition further comprises one or more pharmaceutically acceptable excipients. In one embodiment the composition is administered orally. In another embodiment the composition is enterically coated.

Those of skill in the art will readily appreciate that dose levels can vary as a function of the specific enzyme, the severity of the symptoms and the susceptibility of the subject to side effects. In some embodiments, dosages for a given enzyme are readily determinable by those of skill in the art by a variety of means. An exemplary means is to measure the biological activity of a given compound required to overcome the symptoms.

In one embodiment, the dose of the proteolytic preparation comprising one or more Carica papaya endopeptidases (Enz 1) is 300 mg administered to a subject prior to during or following ingestion of an ATI-containing foodstuff.

The skilled artisan will appreciate that certain factors may influence the dosage and timing required to effectively treat a subject, including the activity of the specific compound employed, the age, body weight, general health, gender, and diet of the subject, the time of administration, the route of administration, the rate of excretion, any drug combination, the degree of expression or activity to be modulated, sensitivity to gluten, previous treatments and other diseases present.

The pharmaceutical compositions can be included in a container, pack, or dispenser together with instructions for administration.

For oral preparations, the compositions according to the present invention can be used alone or in combination with appropriate additives to make tablets, powders, granules or capsules, for example, with conventional additives, such as lactose, mannitol, corn starch or potato starch; with binders, such as crystalline cellulose, cellulose variants, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In one embodiment of the present invention, the oral formulations comprise enteric coatings, so that the active agent is delivered to the intestinal tract. Enteric formulations are often used to protect an active ingredient from the strongly acid contents of the stomach. Such formulations can be created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate, hydroxypropyl methylcellulose acetate succinate, methacrylate copolymers, cellulose acetate phthalate and acrylic coating systems such as Acryl-Ease (Colorcon).

Other enteric formulations comprise engineered polymer microspheres made of biologically erodable polymers, which display strong adhesive interactions with gastrointestinal mucus and cellular linings and can traverse both the mucosal absorptive epithelium and the follicle-associated epithelium covering the lymphoid tissue of Peyer's patches. The polymers maintain contact with intestinal epithelium for extended periods of time and actually penetrate it, through and between cells (see, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71 (3):307-18).

In another aspect the present invention provides a method for digesting an epitope of least one alpha amylase trypsin inhibitor (ATI), the method comprising contacting an ATI comprising the epitope with an effective dose of a composition comprising a proteolytic preparation comprising one or more Carica papaya endopeptidases.

In another embodiment the present invention provides a method as described herein wherein the at least one epitope is selected from;
a) an Alpha-amylase inhibitor precursor (CIII) (WMAI-1) B cell epitope selected from the group consisting of:

AASVPE
ADINNE
ALTGCR
AMVKLQ
AVLRDC
AYPDV
CQQLAD
CRAMVK
CVGSQV
CYGDWA
DCCQQL
ELGVRE
EVMKLT
GCRKEV
GDRAGV
GDWAAY
GKEVLP
GSQVPE
GVCYGD
GVREGK
INNEWC
KVPIPN
LPGCRK
LQCVGS
LRDCCQ
LRSVYQ
LSSMLR
LTAASV
MKLTAA
NEWCRC
PATGYK
PEAVLR
PEVCKV
PSGDRA
QLADIN
QVPEAV
RAGVCY
RCGDLS
REGKEV
RKEVMK
SGPWSW
SMLRSV
SVPEVC
SVYQEL
TGCRAM
TGYKVS
VCKVPI
VKLQCV
VSALTG
WAAYPD
WCRCGD
YKVSAL
YQELGV;

b) a putative alpha-amylase inhibitor B cell epitope selected from the group consisting of PWSWCD, SWCDPA, and SWCDPATGYKVSALTGCRAMV;
c) a peptide comprising an ATI epitope, said peptide selected from the group consisting of:

DCCQQLAHISEWCR
EHGAQEGQAGTGAFPR
CGALYSMLDSMYK
LQCNGSQVPEAVLR
LPIVVDASGDGAYVCK
LTAASITAVCR
SGPWMCYPGQAFQVPALPACRPL LR
DCCQQLADISEWCR
EHGVSEGQAGTGAFPSCR
SGPWMCYPGQAFQVPALPGCRPL LK
ECCQQLADISEWCR
LTAASITAVCK
SGPWMCYPGYAFK
VPALPGCRPVLK;

d) a CM16 and/or CM17 ATI epitope selected from the group consisting of:

IETPGSPYLAK
SDPNSSVLK
ELYDASQHCR
EYVAQQTCGVGIVGSPVSTEPGN TPR
TSDPNSGVLK
VLVTPGHCNVMTVHNTPYCLGLD I
IEMPGPPYLAK
NYVEEQACR
QECCEQLANIPQQCR
SRPDQSGLMELPGCPR
YFMGPK
EVQMDFVR;

e) a monomeric ATI epitope selected from the group consisting of:

EVLPGCR
CGDLSSMLR
DCCQQLADINNEWCR
VSALTGCR
SVYQELGVR
SHNSGPWSWCDPATGYK
LTAASVPEVCK
LQCVGSQVPEAVLR
VPIPNPSGDR
SVYQEIGVR;

f) a CM3 ATI epitope selected from the group consisting of:

LPEWMTSASIYSPGKPYLAK
EMQWDFVR
LYCCQELAEISQQCR
DLPGCPR
LLVAPGQCNLATIHNVR
DYVLQQTCGTFTPGSK
YFIALPVPSQPVDPR
SGNVGESGLIDLPGCPR
LPEWMTSASIFSPMKPYLAK
LYCCQELAEIPQQCR
QMQWDFVR
TDLLPHCR;

g) a CM Hagerman or CM1 ATI epitope selected from the group consisting of:

GPSLPMLVK
SDPNSSVLK;

and
h) a dimeric ATI epitope selected from the group consisting of:

EHGAQEGQAGTGAFPR
EFIAGIVGR.

The method and uses of the present invention can be used for prophylaxis or safeguarding, as well as for therapeutic purposes. Accordingly, as used herein, the term treatment and the like includes any diminution in the severity of a pre-existing disease, condition or symptom of an α-amylase/trypsin inhibitor (ATI) mediated condition, particularly as measured by the severity of symptoms such as, but not limited to, fatigue, chronic diarrhoea, and malabsorption of nutrients, weight loss, abdominal distension, asthma symptoms, anaphylaxis and anaemia. As used herein, the term prophylaxis and the like refer to the prevention of a disease, condition or symptom of an ATI-medicated condition.

Subjects that can benefit from the methods of the present invention may be of any age and include adults and children. Children in particular benefit from prophylactic treatment, as prevention of early exposure to ATI peptides can prevent initial development of the disease. Children suitable for prophylaxis can be identified by genetic testing for predisposition, for example, by HLA typing, by family history, by T cell assay, or by other means known to the skilled addressee.

The methods according to the present invention may also be performed in combination with other modalities, including, but not limited to, administering to a subject in need thereof, an inhibitor of tissue transglutaminase, an anti-inflammatory agent, an anti-ulcer agent, a mast cell-stabilizing agents, and/or and an-allergy agent. Examples of such agents include HMG-CoA reductase inhibitors with anti-inflammatory properties such as compactin, lovastatin, simvastatin, pravastatin and atorvastatin; anti-allergic histamine H1 receptor antagonists such as acrivastine, cetirizine, desloratadine, ebastine, fexofenadine, levocetirizine, loratadine and mizolastine; leukotriene receptor antagonists such as montelukast and zafirlukast; COX2 inhibitors such as celecoxib and rofecoxib; p38 MAP kinase inhibitors such as BIRB-796; and mast cell stabilizing agents such as sodium chromoglycate (chromolyn), pemirolast, proxicromil, repirinast, doxantrazole, amlexanox nedocromil and probicromil.

Various methods for administration may be employed, preferably using oral administration, for example with meals. The dosage of the therapeutic formulation will vary widely, depending upon the nature of the disease, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, with meals, semi-weekly, or otherwise as needed to maintain an effective dosage level.

The therapeutic effect can be measured in terms of clinical outcome or can be determined by immunological or biochemical tests. Alternatively, one can look for a reduction in severity of the symptoms of the disease.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention before the priority date of each claim of this application.

Finally it is to be understood that various other modifications and/or alterations may be made without departing from the spirit of the present invention as outlined herein.

Certain embodiments of the present invention will now be described in the following examples. It should be understood, however, that the following description is illustrative only and should not be taken in any way as a restriction on the generality of the invention described above.

### EXAMPLES

### Example 1: ATI Isolation and Purification

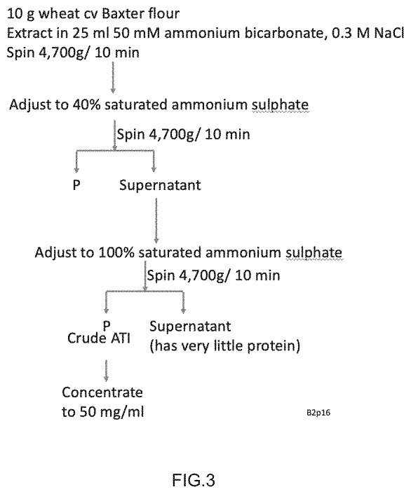

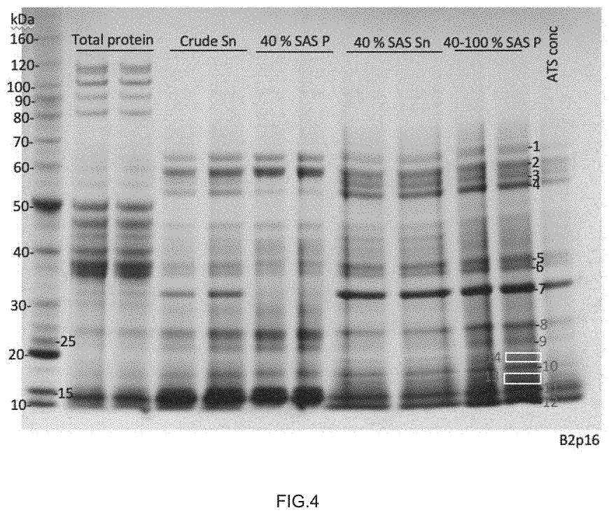

A crude preparation of wheat cv Baxter ATIs was made following the method of Zevallos (2017) Gastroenterology 152, 1103 (FIG. 3, FIG. 4).

Increasing concentrations of ammonium sulphate were used to selectively “salt out” amylase trypsin inhibitors from wheat cv Baxter flour. The final concentrate (ATIs conc) has the same banding pattern as the proteins in the 40-100% saturated ammonium sulphate pellet (40-100% SASP), confirming that all proteins were effectively concentrated and there was no loss during concentration using centrifugal concentration with 5 kDa filter.

The same banding pattern is seen in the 40% saturated ammonium sulphate supernatant (40 SAS Sn) but not the 40% saturated ammonium sulphate pellet (40 SAS P), confirming a subset of proteins was being concentrated by the additional ammonium sulphate. These protein bands are only minor components of the crude extract (Crude Sn) and difficult to observe in the total protein extract (Total protein) which was made by dissolving all proteins in the aggressive solvent, 8M urea, 1% DTT, 20 mM triethylamine buffer.

This showed the final preparation was enriched for particular bands of the correct molecular weight to be ATIs. These bands were cut from the gel and identified by protein sequencing (Table 1). There were no ATIs present in bands 1 to 7. Various ATI types were detected in samples 8 to 14. They were among the top 10 to 20 hits in sample 10, 11, 12 and 13, indicating the relative ATI concentration in these gel bands was high.

TABLE 1
Identification of ATI proteins in gel slices

| Protein Band | MW (kDa) | ATI proteins |
|---|---|---|
| 8 | 27.2 | 0.19 dimeric alpha-amylase inhibitor |
| 9 | 22.7 | 0.19 dimeric alpha-amylase inhibitor |
| | | endogenous alpha-amylase/subtilisin inhibitor |
| | | monomeric alpha-amylase inhibitor |
| | | alpha amylase inhibitor CM3 |
| 14 | ~20 | alpha amylase inhibitor CM3 |
| | | 0.19 dimeric alpha-amylase inhibitor |
| | | monomeric alpha-amylase inhibitor |
| | | alpha-amylase inhibitor CM16 subunit |
| 10 | 17.3 | alpha amylase inhibitor CM3 |
| | | 0.19 dimeric alpha-amylase inhibitor |
| | | monomeric alpha-amylase inhibitor |
| | | alpha-amylase inhibitor CM16 subunit |
| | | alpha-amylase/trypsin inhibitor CM2 |
| 13 | ~15 | alpha amylase inhibitor CM3 |
| | | monomeric alpha-amylase inhibitor |
| | | 0.19 dimeric alpha-amylase inhibitor |
| | | alpha-amylase inhibitor CM16 subunit |
| | | alpha-amylase/trypsin inhibitor CM1 |
| | | alpha-amylase/trypsin inhibitor CM3 |
| | | dimeric alpha-amylase inhibitor |
| | | alpha-amylase/trypsin inhibitor CM2 |
| 11 | 12.8 | 0.19 dimeric alpha-amylase inhibitor |
| | | monomeric alpha-amylase inhibitor |
| | | alpha amylase inhibitor CM1 |
| | | alpha amylase inhibitor CM3 |
| | | dimeric alpha-amylase inhibitor |
| | | alpha-amylase inhibitor CM16 subunit |
| | | putative alpha-amylase inhibitor CM2 |
| | | PUP88 protein trypsin/alpha-amylase inhibitors |
| | | trypsin/alpha-amylase inhibitor CMX1/CMX3 |
| 12 | 9.0 | monomeric alpha-amylase inhibitor |
| | | alpha amylase inhibitor CM3 |
| | | 0.19 dimeric alpha-amylase inhibitor |
| | | alpha-amylase inhibitor CM16 subunit |
| | | alpha-amylase/trypsin inhibitor CM2 |
| | | trypsin/alpha-amylase inhibitor CMX1/CMX3 |
| | | alpha-amylase/trypsin inhibitor CM1 |

There appears to be considerable cross-contamination of excised protein bands by ATI species (e.g. CM3 is present in all bands cut from 9.0-22.7 kDa). This is due to cross-linking of different species by internal—S—S— bonds, resistant to reduction by DTT prior to electrophoresis.

A proteolytic preparation comprising Carica papaya endopeptidases was used in the Examples. As demonstrated in example 8, the Enz 1 preparation comprising Carica papaya endopeptidases comprises the enzymes caricain, multiple isoforms of papain, chymopapain and papaya proteinase 4.

### Example 2: ATI Digestion by a Proteolytic Preparation Comprising Carica papaya Endopeptidases

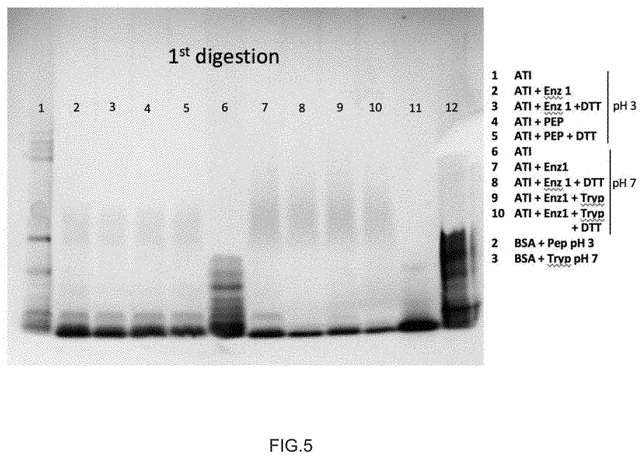

A composition comprising a proteolytic preparation comprising Carica papaya endopeptidases (at 1× concentration) was added to ATI concentrate plus or minus DTT in the presence of pepsin or trypsin. Importantly, all ATI bands were completely dissolved (FIG. 5), by a proteolytic preparation comprising Carica papaya endopeptidases.

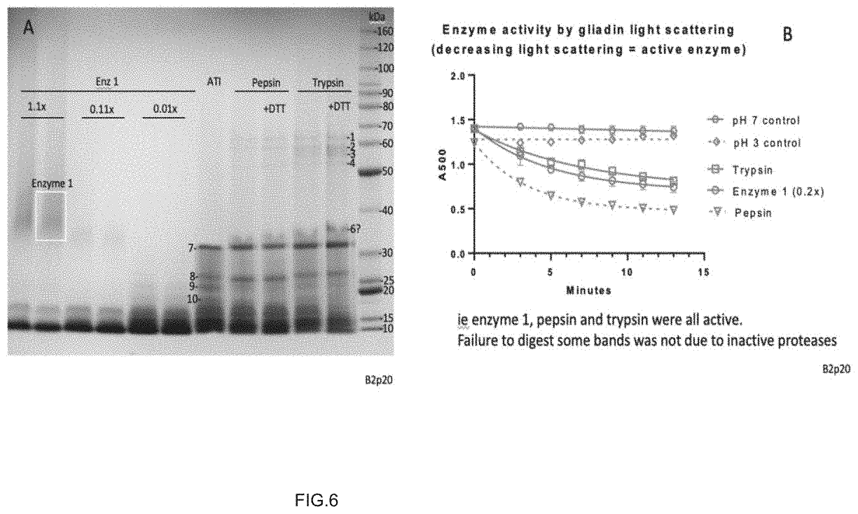

ATI proteins (964 μg) corresponding to ˜10 g wheat in 100 mL stomach, were digested with the proteolytic preparation comprising caricain (Enzyme 1; “Enz1”) at 1.1×concentration (i.e. 1×300 mg pill of Enz1 proteolytic preparation in 100 mL stomach). Every fraction with added the proteolytic preparation comprising caricain, has completely digested all putative ATI protein bands. The addition of DTT, pepsin or trypsin had no effect as the digestion was complete in all cases. The control lanes (ATI, lane 6 and 12, but not lane 1) also show some hydrolysis. This is also seen in some later experiments, probably because small amounts of the very active Enzyme 1 composition has migrated from neighbouring lanes of the gel during electrophoresis. Enzyme activity of all enzymes was confirmed by light scattering (see FIG. 6B).

### Example 3: ATI Digestion by a Proteolytic Preparation Comprising Carica papaya Endopeptidases (Enz 1), and Resistance of ATI to Digestion with Pepsin and Trypsin

Hydrolysis of ATIs by a proteolytic preparation comprising Carica papaya endopeptidases (‘Enzyme 1’ or ‘Enz 1’) at various concentrations (1.1×, 0.11×, and 0.011×) was compared to digestion/hydrolysis by pepsin and trypsin plus or minus dithiothreitol (DTT). All bands were digested by Enzyme 1 but few bands were digested by pepsin or trypsin; indicating that the bands not digested by pepsin or trypsin were ATI proteins which are resistant to these proteases (FIG. 6).

The reducing agent, DTT was added to some reactions. DTT cleaves disulphide (S—S) linkages which are numerous in ATIs and hold the tight conformation which confers proteolytic resistance on the ATI proteins. DTT did not speed proteolysis of putative ATI bands, confirming the pepsin/trypsin resistance.

Enzyme activity was followed by SDS-PAGE (A) or by light scattering (B). The proteolytic preparation comprising caricain, was added at 10× increasing concentrations from 0.011×, 0.11× and 1.11× corresponding to enzyme protein amounts in the final assay of 0.24 μg, 2.4 μg and 24 μg, respectively. These concentrations completely hydrolysed all putative ATI bands. At ˜1,000 μg ATI protein, this corresponds to an Enzyme 1: protein (ATI) ratio of 1:10,000), well below the amount of protease: protein normally added for complete digestion (1:100) confirming the efficient activity of the Enzyme 1 preparation. On the other hand, pepsin and trypsin did not digest band 1, 2, 3, 4, 7 or 8 even when added at a ratio of 1:100. The addition of DTT did not accelerate the pepsin/trypsin digestion.

Light scattering confirmed in this and all subsequent experiments showed that Enzyme 1, pepsin and trypsin were active and capable of hydrolysing gliadin when assayed by light scattering (FIG. 6B). In brief, gliadin precipitates when added to an aqueous buffer. As proteolysis occurs the solution clarifies as the gliadin is hydrolysed into small soluble fragments. This process can be followed by monitoring the decrease in absorbance produced by the reduced light scattering. The absorbance of the Enzyme 1, pepsin and trypsin treated gliadin all decrease, confirming enzyme activity, whereas the absorbance of the pH 3 and pH 7 controls do not decrease.

### Example 4: ATI Digestion by a Proteolytic Preparation Comprising Carica papaya Endopeptidases

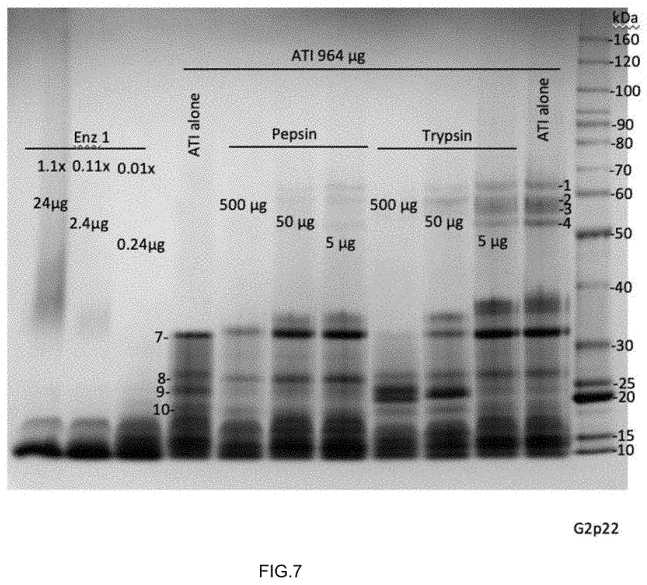

To assess whether insufficient pepsin and trypsin was added to previous digestions, the effect of higher amounts of pepsin and trypsin was examined. Hydrolysis by different amounts of the proteolytic preparation comprising caricain, (24, 2.4, 0.24 μg per reaction), was compared to hydrolysis of different amounts of pepsin and trypsin (500, 50, 5 μg) (FIG. 7). All bands were digested by the proteolytic preparation a proteolytic preparation comprising Carica papaya endopeptidases (‘Enz 1’), even at the lowest concentration, whereas pepsin and trypsin did not digest key bands at a concentration at 5 ug, 10× higher than Enz 1. Digestion of key bands was not observed at 50 ug and 500 ug pepsin or trypsin—indicating that a 2,000 excess of these proteases was ineffective.

Digestion using ten-fold reductions in the amount of the proteolytic preparation comprising Carica papaya endopeptidases (Enz 1) from 24, 2.4 or 0.24 μg on ˜1,000 μg of ATI was compared to the digestion of ATIs using decreasing amounts of pepsin or trypsin (500, 50 or 0.5 μg). All bands in the ATI preparation were digested by the proteolytic preparation comprising Carica papaya endopeptidases (Enz 1), even at the lowest concentration, whereas pepsin and trypsin did not digest key bands at a concentration at 5 ug, 10×higher than the Enzyme 1 preparation. Digestion of key ATI bands was not observed at 50 ug and 500 ug pepsin or trypsin—indicating that a 2,000×excess of these proteases was ineffective compared to the proteolytic preparation comprising caricain. The ATI alone standard, in lane 4, exhibited some proteolysis due to contamination from neighbouring lanes, however the ‘ATI alone’ standard in lane 11 did not—due to the absence of an active enzyme in the neighbouring lanes, confirming this assumption.

### Example 5: ATI Digestion by a Proteolytic Preparation Comprising Carica papaya Endopeptidases

The ability of a proteolytic preparation comprising Carica papaya endopeptidases to digest B cell and TLR4 epitopes of ATIs was examined.

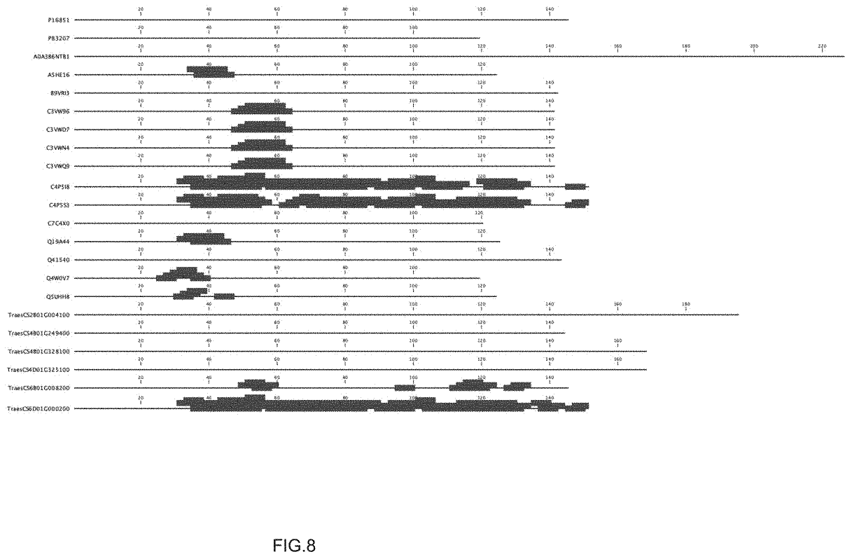

In brief, ATIs were treated with pepsin followed by trypsin (approx. 50 mg/ml) in the presence of or absence of a preparation comprising Carica papaya endopeptidases, both at pH 7 or pH 3. The resulting peptides were mapped to the ATI proteins identified in the samples. In particular, the resulting peptides were mapped to ATI proteins and overlap with baker's asthma related B cell epitopes (FIG. 8, Table 2) was examined to determine if immunogenic epitopes remained following digestion with enzymes.

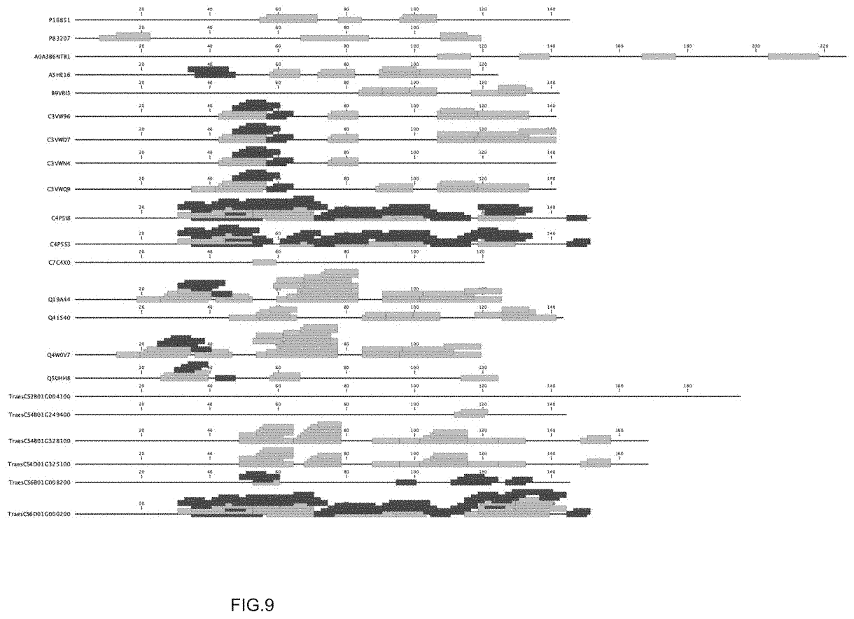

FIG. 9 shows that peptides detected from digests using pepsin followed by trypsin in the absence of a proteolytic preparation comprising Carica papaya endopeptidases (Enz 1) at pH 7, overlap with ATI B cell epitopes. Importantly, there were no peptides detected from digests using pepsin followed by trypsin in the presence of a proteolytic preparation comprising Carica papaya endopeptidases (Enz 1) at pH 7, indicating that following digestion with pepsin and trypsin in the presence of a proteolytic preparation comprising Carica papaya endopeptidases (Enz 1), immunogenic B cell epitopes of ATIs are completely digested. This indicates that following digestion with pepsin and trypsin in the gastrointestinal tract immunogenic epitopes of ATIs remain intact and are therefore available to induce intestinal and extraintestinal symptoms.

Notably, peptides comprising the immunogenic epitopes of Table 2 were not detected following

TABLE 2
B cell epitopes of Triticum aestivum ATIs mapped in Example 5

| | | Epi- | Epi- | | |
|---|---|---|---|---|---|
| | | tope | tope | | |
| | | Start- | End- | | |
| | | ing | ing | | |
| Epitope | Epitope | Posi- | Posi- | Epitope | Epitope |
| Epitope IRI | Description | tion | tion | Antigen Name | Antigen IRI |
| http://www.iedb.org/ | AASVPE | 121 | 126 | Alpha-amylase | http://www.ncbi.nlm.nih.gov/ |
| epitope/450 | | | | inhibitor 0.28 | protein/P01083.3 |
| | | | | precursor (CIII) | |
| | | | | (WMAI-1) | |
| http://www.iedb.org/ | ADINNE | 77 | 82 | Alpha-amylase | http://www.ncbi.nlm.nih.gov/ |
| epitope/688 | | | | inhibitor 0.28 | protein/P01083.3 |
| | | | | precursor (CIII) | |
| | | | | (WMAI-1) | |
| http://www.iedb.org/ | ALTGCR | 47 | 52 | Alpha-amylase | http://www.ncbi.nlm.nih.gov/ |
| epitope/2926 | | | | inhibitor 0.28 | protein/P01083.3 |
| | | | | precursor (CIII) | |
| | | | | (WMAI-1) | |
| http://www.iedb.org/ | AMVKLQ | 53 | 58 | Alpha-amylase | http://www.ncbi.nlm.nih.gov/ |
| epitope/3204 | | | | inhibitor 0.28 | protein/P01083.3 |
| | | | | precursor (CIII) | |
| | | | | (WMAI-1) | |
| http://www.iedb.org/ | AVLRDC | 67 | 72 | Alpha-amylase | http://www.ncbi.nlm.nih.gov/ |
| epitope/5450 | | | | inhibitor 0.28 | protein/P01083.3 |
| | | | | precursor (CIII) | |
| | | | | (WMAI-1) | |
| http://www.iedb.org/ | AYPDV | 149 | 153 | Alpha-amylase | http://www.ncbi.nlm.nih.gov/ |
| epitope/5890 | | | | inhibitor 0.28 | protein/P01083.3 |
| | | | | precursor (CIII) | |
| | | | | (WMAI-1) | |
| http://www.iedb.org/ | CQQLAD | 73 | 78 | Alpha-amylase | http://www.ncbi.nlm.nih.gov/ |
| epitope/6893 | | | | inhibitor 0.28 | protein/P01083.3 |
| | | | | precursor (CIII) | |
| | | | | (WMAI-1) | |
| http://www.iedb.org/ | CRAMVK | 51 | 56 | Alpha-amylase | http://www.ncbi.nlm.nih.gov/ |
| epitope/6909 | | | | inhibitor 0.28 | protein/P01083.3 |
| | | | | precursor (CIII) | |
| | | | | (WMAI-1) | |
| http://www.iedb.org/ | CVGSQV | 59 | 64 | Alpha-amylase | http://www.ncbi.nlm.nih.gov/ |
| epitope/7268 | | | | inhibitor 0.28 | protein/P01083.3 |
| | | | | precursor (CIII) | |
| | | | | (WMAI-1) | |
| http://www.iedb.org/ | CYGDWA | 143 | 148 | Alpha-amylase | http://www.ncbi.nlm.nih.gov/ |
| epitope/7367 | | | | inhibitor 0.28 | protein/P01083.3 |
| | | | | precursor (CIII) | |
| | | | | (WMAI-1) | |
| http://www.iedb.org/ | DCCQQL | 71 | 76 | Alpha-amylase | http://www.ncbi.nlm.nih.gov/ |
| epitope/7698 | | | | inhibitor 0.28 | protein/P01083.3 |
| | | | | precursor (CIII) | |
| | | | | (WMAI-1) | |
| http://www.iedb.org/ | ELGVRE | 99 | 104 | Alpha-amylase | http://www.ncbi.nlm.nih.gov/ |
| epitope/13084 | | | | inhibitor 0.28 | protein/P01083.3 |
| | | | | precursor (CIII) | |
| | | | | (WMAI-1) | |
| http://www.iedb.org/ | EVMKLT | 115 | 120 | Alpha-amylase | http://www.ncbi.nlm.nih.gov/ |
| epitope/14826 | | | | inhibitor 0.28 | protein/P01083.3 |
| | | | | precursor (CIII) | |
| | | | | (WMAI-1) | |
| http://www.iedb.org/ | GCRKEV | 111 | 116 | Alpha-amylase | http://www.ncbi.nlm.nih.gov/ |
| epitope/18906 | | | | inhibitor 0.28 | protein/P01083.3 |
| | | | | precursor (CIII) | |
| | | | | (WMAI-1) | |
| http://www.iedb.org/ | GDRAGV | 137 | 142 | Alpha-amylase | http://www.ncbi.nlm.nih.gov/ |
| epitope/19100 | | | | inhibitor 0.28 | protein/P01083.3 |
| | | | | precursor (CIII) | |
| | | | | (WMAI-1) | |
| http://www.iedb.org/ | GDWAAY | 145 | 150 | Alpha-amylase | http://www.ncbi.nlm.nih.gov/ |
| epitope/19179 | | | | inhibitor 0.28 | protein/P01083.3 |
| | | | | precursor (CIII) | |
| | | | | (WMAI-1) | |
| http://www.iedb.org/ | GKEVLP | 105 | 110 | Alpha-amylase | http://www.ncbi.nlm.nih.gov/ |
| epitope/20555 | | | | inhibitor 0.28 | protein/P01083.3 |
| | | | | precursor (CIII) | |
| | | | | (WMAI-1) | |
| http://www.iedb.org/ | GSQVPE | 61 | 66 | Alpha-amylase | http://www.ncbi.nlm.nih.gov/ |
| epitope/22475 | | | | inhibitor 0.28 | protein/P01083.3 |
| | | | | precursor (CIII) | |
| | | | | (WMAI-1) | |
| http://www.iedb.org/ | GVCYGD | 141 | 146 | Alpha-amylase | http://www.ncbi.nlm.nih.gov/ |
| epitope/22929 | | | | inhibitor 0.28 | protein/P01083.3 |
| | | | | precursor (CIII) | |
| | | | | (WMAI-1) | |
| http://www.iedb.org/ | GVREGK | 101 | 106 | Alpha-amylase | http://www.ncbi.nlm.nih.gov/ |
| epitope/23121 | | | | inhibitor 0.28 | protein/P01083.3 |
| | | | | precursor (CIII) | |
| | | | | (WMAI-1) | |
| http://www.iedb.org/ | INNEWC | 79 | 84 | Alpha-amylase | http://www.ncbi.nlm.nih.gov/ |
| epitope/27693 | | | | inhibitor 0.28 | protein/P01083.3 |
| | | | | precursor (CIII) | |
| | | | | (WMAI-1) | |
| http://www.iedb.org/ | KVPIPN | 129 | 134 | Alpha-amylase | http://www.ncbi.nlm.nih.gov/ |
| epitope/34158 | | | | inhibitor 0.28 | protein/P01083.3 |
| | | | | precursor (CIII) | |
| | | | | (WMAI-1) | |
| http://www.iedb.org/ | LPGCRK | 109 | 114 | Alpha-amylase | http://www.ncbi.nlm.nih.gov/ |
| epitope/38493 | | | | inhibitor 0.28 | protein/P01083.3 |
| | | | | precursor (CIII) | |
| | | | | (WMAI-1) | |
| http://www.iedb.org/ | LQCVGS | 57 | 62 | Alpha-amylase | http://www.ncbi.nlm.nih.gov/ |
| epitope/38813 | | | | inhibitor 0.28 | protein/P01083.3 |
| | | | | precursor (CIII) | |
| | | | | (WMAI-1) | |
| http://www.iedb.org/ | LRDCCQ | 69 | 74 | Alpha-amylase | http://www.ncbi.nlm.nih.gov/ |
| epitope/39046 | | | | inhibitor 0.28 | protein/P01083.3 |
| | | | | precursor (CIII) | |
| | | | | (WMAI-1) | |
| http://www.iedb.org/ | LRSVYQ | 93 | 98 | Alpha-amylase | http://www.ncbi.nlm.nih.gov/ |
| epitope/39217 | | | | inhibitor 0.28 | protein/P01083.3 |
| | | | | precursor (CIII) | |
| | | | | (WMAI-1) | |
| http://www.iedb.org/ | LSSMLR | 89 | 94 | Alpha-amylase | http://www.ncbi.nlm.nih.gov/ |
| epitope/39673 | | | | inhibitor 0.28 | protein/P01083.3 |
| | | | | precursor (CIII) | |
| | | | | (WMAI-1) | |
| http://www.iedb.org/ | LTAASV | 119 | 124 | Alpha-amylase | http://www.ncbi.nlm.nih.gov/ |
| epitope/39782 | | | | inhibitor 0.28 | protein/P01083.3 |
| | | | | precursor (CIII) | |
| | | | | (WMAI-1) | |
| http://www.iedb.org/ | MKLTAA | 117 | 122 | Alpha-amylase | http://www.ncbi.nlm.nih.gov/ |
| epitope/41862 | | | | inhibitor 0.28 | protein/P01083.3 |
| | | | | precursor (CIII) | |
| | | | | (WMAI-1) | |
| http://www.iedb.org/ | NEWCRC | 81 | 86 | Alpha-amylase | http://www.ncbi.nlm.nih.gov/ |
| epitope/43791 | | | | inhibitor 0.28 | protein/P01083.3 |
| | | | | precursor (CIII) | |
| | | | | (WMAI-1) | |
| http://www.iedb.org/ | PATGYK | 39 | 44 | Alpha-amylase | http://www.ncbi.nlm.nih.gov/ |
| epitope/46967 | | | | inhibitor 0.28 | protein/P01083.3 |
| | | | | precursor (CIII) | |
| | | | | (WMAI-1) | |
| http://www.iedb.org/ | PEAVLR | 65 | 70 | Alpha-amylase | http://www.ncbi.nlm.nih.gov/ |
| epitope/47240 | | | | inhibitor 0.28 | protein/P01083.3 |
| | | | | precursor (CIII) | |
| | | | | (WMAI-1) | |
| http://www.iedb.org/ | PEVCKV | 125 | 130 | Alpha-amylase | http://www.ncbi.nlm.nih.gov/ |
| epitope/47446 | | | | inhibitor 0.28 | protein/P01083.3 |
| | | | | precursor (CIII) | |
| | | | | (WMAI-1) | |
| http://www.iedb.org/ | PSGDRA | 135 | 140 | Alpha-amylase | http://www.ncbi.nlm.nih.gov/ |
| epitope/49372 | | | | inhibitor 0.28 | protein/P01083.3 |
| | | | | precursor (CIII) | |
| | | | | (WMAI-1) | |
| http://www.iedb.org/ | PWSWCD | 3 | 8 | putative alpha- | http://www.ncbi.nlm.nih.gov/ |
| epitope/50052 | | | | amylase | protein/CBA13560.1 |
| | | | | inhibitor 0.28 | |
| http://www.iedb.org/ | QLADIN | 75 | 80 | Alpha-amylase | http://www.ncbi.nlm.nih.gov/ |
| epitope/51279 | | | | inhibitor 0.28 | protein/P01083.3 |
| | | | | precursor (CIII) | |
| | | | | (WMAI-1) | |
| http://www.iedb.org/ | QVPEAV | 63 | 68 | Alpha-amylase | http://www.ncbi.nlm.nih.gov/ |
| epitope/52755 | | | | inhibitor 0.28 | protein/P01083.3 |
| | | | | precursor (CIII) | |
| | | | | (WMAI-1) | |
| http://www.iedb.org/ | RAGVCY | 139 | 144 | Alpha-amylase | http://www.ncbi.nlm.nih.gov/ |
| epitope/53109 | | | | inhibitor 0.28 | protein/P01083.3 |
| | | | | precursor (CIII) | |
| | | | | (WMAI-1) | |
| http://www.iedb.org/ | RCGDLS | 85 | 90 | Alpha-amylase | http://www.ncbi.nlm.nih.gov/ |
| epitope/53273 | | | | inhibitor 0.28 | protein/P01083.3 |
| | | | | precursor (CIII) | |
| | | | | (WMAI-1) | |
| http://www.iedb.org/ | REGKEV | 103 | 108 | Alpha-amylase | http://www.ncbi.nlm.nih.gov/ |
| epitope/53509 | | | | inhibitor 0.28 | protein/P01083.3 |
| | | | | precursor (CIII) | |
| | | | | (WMAI-1) | |
| http://www.iedb.org/ | RKEVMK | 113 | 118 | Alpha-amylase | http://www.ncbi.nlm.nih.gov/ |
| epitope/54340 | | | | inhibitor 0.28 | protein/P01083.3 |
| | | | | precursor (CIII) | |
| | | | | (WMAI-1) | |
| http://www.iedb.org/ | SGPWSW | 31 | 36 | Alpha-amylase | http://www.ncbi.nlm.nih.gov/ |
| epitope/58206 | | | | inhibitor 0.28 | protein/P01083.3 |
| | | | | precursor (CIII) | |
| | | | | (WMAI-1) | |
| http://www.iedb.org/ | SMLRSV | 91 | 96 | Alpha-amylase | http://www.ncbi.nlm.nih.gov/ |
| epitope/59703 | | | | inhibitor 0.28 | protein/P01083.3 |
| | | | | precursor (CIII) | |
| | | | | (WMAI-1) | |
| http://www.iedb.org/ | SVPEVC | 123 | 128 | Alpha-amylase | http://www.ncbi.nlm.nih.gov/ |
| epitope/62277 | | | | inhibitor 0.28 | protein/P01083.3 |
| | | | | precursor (CIII) | |
| | | | | (WMAI-1) | |
| http://www.iedb.org/ | SVYQEL | 95 | 100 | Alpha-amylase | http://www.ncbi.nlm.nih.gov/ |
| epitope/62417 | | | | inhibitor 0.28 | protein/P01083.3 |
| | | | | precursor (CIII) | |
| | | | | (WMAI-1) | |
| http://www.iedb.org/ | SWCDPA | 5 | 10 | putative alpha- | http://www.ncbi.nlm.nih.gov/ |
| epitope/62429 | | | | amylase | protein/CBA13560.1 |
| | | | | inhibitor 0.28 | |
| http://www.iedb.org/ | SWCDPATGYKVS | 5 | 25 | putative alpha- | http://www.ncbi.nlm.nih.gov/ |
| epitope/62430 | ALTGCRAMV | | | amylase | protein/CBA13560.1 |
| | | | | inhibitor 0.28 | |
| http://www.iedb.org/ | TGCRAM | 49 | 54 | Alpha-amylase | http://www.ncbi.nlm.nih.gov/ |
| epitope/63798 | | | | inhibitor 0.28 | protein/P01083.3 |
| | | | | precursor (CIII) | |
| | | | | (WMAI-1) | |
| http://www.iedb.org/ | TGYKVS | 41 | 46 | Alpha-amylase | http://www.ncbi.nlm.nih.gov/ |
| epitope/64007 | | | | inhibitor 0.28 | protein/P01083.3 |
| | | | | precursor (CIII) | |
| | | | | (WMAI-1) | |
| http://www.iedb.org/ | VCKVPI | 127 | 132 | Alpha-amylase | http://www.ncbi.nlm.nih.gov/ |
| epitope/67859 | | | | inhibitor 0.28 | protein/P01083.3 |
| | | | | precursor (CIII) | |
| | | | | (WMAI-1) | |
| http://www.iedb.org/ | VKLQCV | 55 | 60 | Alpha-amylase | http://www.ncbi.nlm.nih.gov/ |
| epitope/69261 | | | | inhibitor 0.28 | protein/P01083.3 |
| | | | | precursor (CIII) | |
| | | | | (WMAI-1) | |
| http://www.iedb.org/ | VSALTG | 45 | 50 | Alpha-amylase | http://www.ncbi.nlm.nih.gov/ |
| epitope/70881 | | | | inhibitor 0.28 | protein/P01083.3 |
| | | | | precursor (CIII) | |
| | | | | (WMAI-1) | |
| http://www.iedb.org/ | WAAYPD | 147 | 152 | Alpha-amylase | http://www.ncbi.nlm.nih.gOv/p |
| epitope/72215 | | | | inhibitor 0.28 | protein/P01083.3 |
| | | | | precursor (CIII) | |
| | | | | (WMAI-1) | |
| http://www.iedb.org/ | WCRCGD | 83 | 88 | Alpha-amylase | http://www.ncbi.nlm.nih.gov/ |
| epitope/72280 | | | | inhibitor 0.28 | protein/P01083.3 |
| | | | | precursor (CIII) | |
| | | | | (WMAI-1) | |
| http://www.iedb.org/ | YKVSAL | 43 | 48 | Alpha-amylase | http://www.ncbi.nlm.nih.gov/ |
| epitope/74533 | | | | inhibitor 0.28 | protein/P01083.3 |
| | | | | precursor (CIII) | |
| | | | | (WMAI-1) | |
| http://www.iedb.org/ | YQELGV | 97 | 102 | Alpha-amylase | http://www.ncbi.nlm.nih.gov/ |
| epitope/75479 | | | | inhibitor 0.28 | protein/P01083.3 |
| | | | | precursor (CIII) | |
| | | | | (WMAI-1) | |

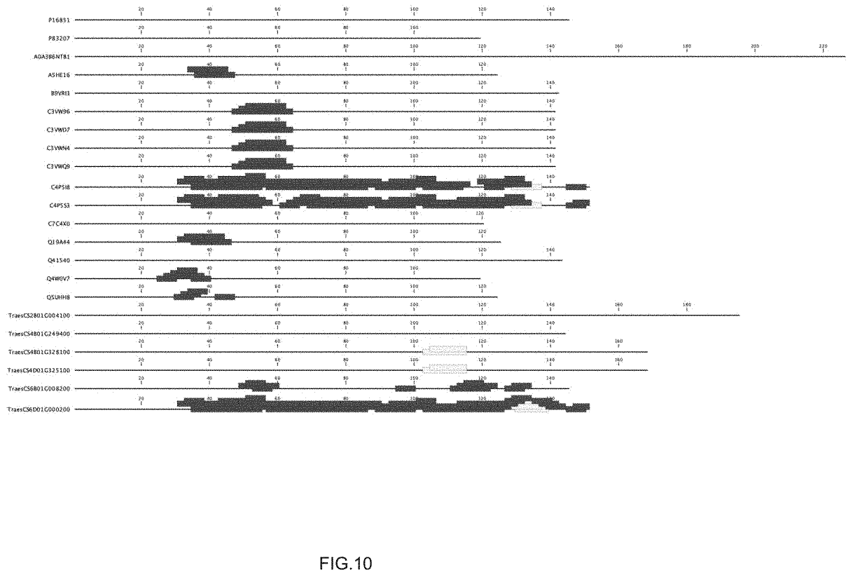

FIG. 10 shows few peptides were detected from digests using pepsin followed by trypsin in the presence of a proteolytic preparation comprising Carica papaya endopeptidases (Enz 1) at pH 7, indicating that following digestion with pepsin and trypsin in the presence of a proteolytic preparation comprising Carica papaya endopeptidases (Enz 1), almost all immunogenic B cell epitopes of ATIs are digested.

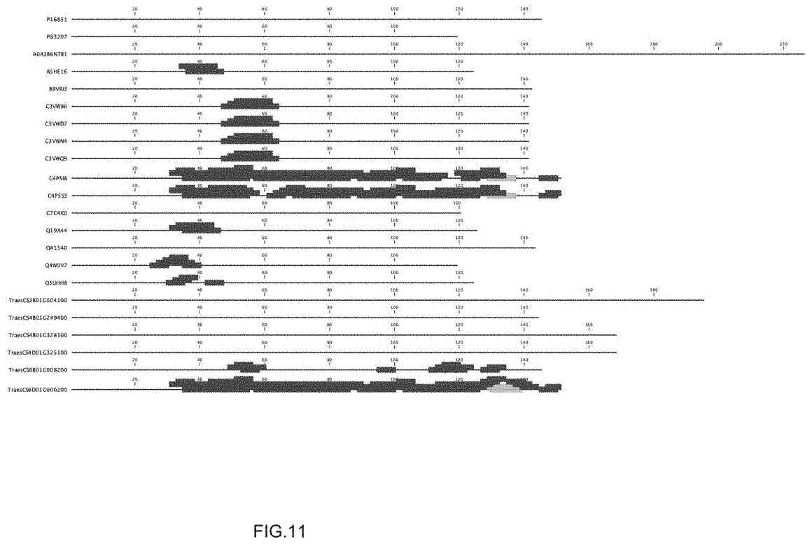

FIG. 11 shows few peptides were detected from digests using pepsin followed by trypsin in the presence of a proteolytic preparation comprising Carica papaya endopeptidases (Enz 1) at pH 3, indicating that following digestion with pepsin and trypsin in the presence of a proteolytic preparation comprising Carica papaya endopeptidases (Enz 1), almost all immunogenic B cell epitopes of ATIs are digested.

### Example 6: ATI Digestion by a Proteolytic Preparation Comprising Carica papaya Endopeptidases

The ability of a proteolytic preparation comprising Carica papaya endopeptidases to digest epitopes of ATIs was examined.

In brief, two commercial wheat cultivars, Baxter and Lancer were used in the analyses. Grain samples were ground into fine powder and defatted using n-pentane. A proteolytic preparation comprising Carica papaya endopeptidases was dissolved in 9 ml pH3 buffered Ammonium Bicarbonate (AmBic) solution. The suspension was centrifuged and further diluted using a final enzyme concentration of 0.11×. Proteins were extracted using isopropanol (IPA) in presence of 2% DTT. Protein extract (100 ul) was applied to a 3 kDa MWCO filter (Millipore) and filter digested using either 1:50 protein-to-enzyme ratio of modified Trypsin (Promega, sequencing grade) or chymotrypsin (Promega, sequencing grade); 0.11×caricain (a proteolytic preparation comprising Carica papaya endopeptidases) or combination of enzymes according to the following experimental design.

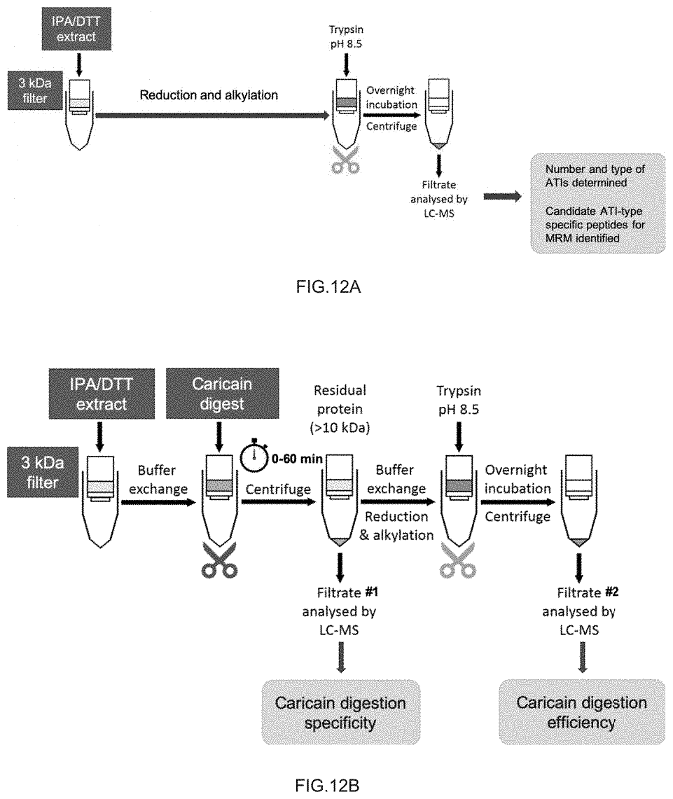

A schematic of Experiment 1 is shown in FIG. 12A. In Experiment 1, 100 ul of IPA/DTT extracts were applied to the filters and alkylated using 50 mM iodoacetamide followed by an overnight digestion using sequencing grade modified trypsin (Promega) or chymotrypsin (Promega). Digested peptides were washed with AmBic, centrifuged and lyophilized. Samples were resuspended in 100 ul of 1% formic acid and subjected to information dependent data acquisition (IDA) using an Ekspert nanoLC415 (Eksigent) coupled to a TripleTOF 6600 MS (SCIEX) instrument. Protein Pilot software v. 5.0.3 and Paragon algorithm (SCIEX) was used for the protein identification. Obtained spectral libraries were searched against in silico developed tryptic or chymotryptic spectral library of Triticeae proteins collected from the Uniprot database (version May 2019) appended with translated gene models obtained from high resolution genome sequencing data of Triticeae species (Ensembl plants). ATI proteins were precisely identified using conserved protein domain information and manually aligned and analysed for sub-type annotation. Fully tryptic or chymotryptic, intense ATI peptides were used to develop multiple reaction monitoring (MRM) methods. MRM transitions were determined for each peptide using the precursor ion and fragment ion m/z values obtained from the IDA analysis. Pooled samples of the trypsin or chymotrypsin digests were separated on an Exion LC system (SCIEX) and analysed on a 6500 QTRAP MS instrument (SCIEX). At least three peptides and four transitions were considered for each protein. From these, three intense transitions with matching peak shape and retention time values were selected for scheduled MRM analysis. Peaks were integrated using Skyline software package (Pino et al., 2017). The peak areas of the transitions obtained from the same peptide were summed and replicate values were subjected to statistical analysis using Graph Pad Prism 8.

A schematic of Experiment 2 is shown in FIG. 12B. In Experiment 2, a two-step digestion was used. First, protein extracts were applied to the 3 kDa filter and digested with the proteolytic preparation comprising Carica papaya endopeptidases using the solution as described above. Aliquots were taken 5, 15, 30, 45 or 60 minutes after digestion with the samples were centrifuged and Filtrate 1 was collected for digestion specificity analysis. The residual protein was further digested by trypsin after buffer exchange (to pH 8.5) followed by reduction and alkylation steps. After the overnight digestion step peptides were collected by centrifugation, washed with AmBic and lyophilized. Samples were reconstituted in 1% FA and subjected to scheduled MRM analysis.

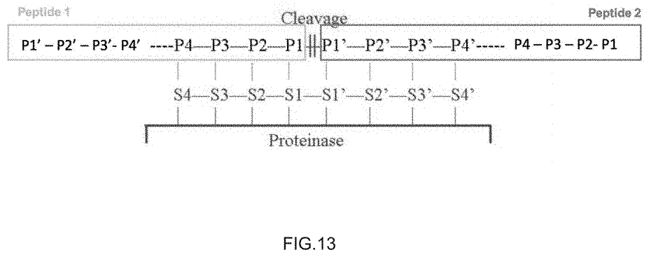

Digestion specificity of the proteolytic preparation comprising Carica papaya endopeptidases was determined by a detailed sequence analysis using the Filtrate 1 peptide results. First peptide sequences obtained from a combined search of Baxter and Lancer extracts using the 5 to 60 minutes samples were analysed together. First three (P′1, P′2 and P′3) and last three (P3, P2, P1) amino acid residues (FIG. 13) were determined for each peptide and frequency of P′1-P1 and P′2P1′-P1P2 pairs calculated. Amino acids were grouped based on their physicochemical features (aliphatic, aromatic, acidic, basic, hydroxylic, sulphur-containing and amidic) and frequency calculations were performed both at amino acid group and individual amino acid-pairs levels.

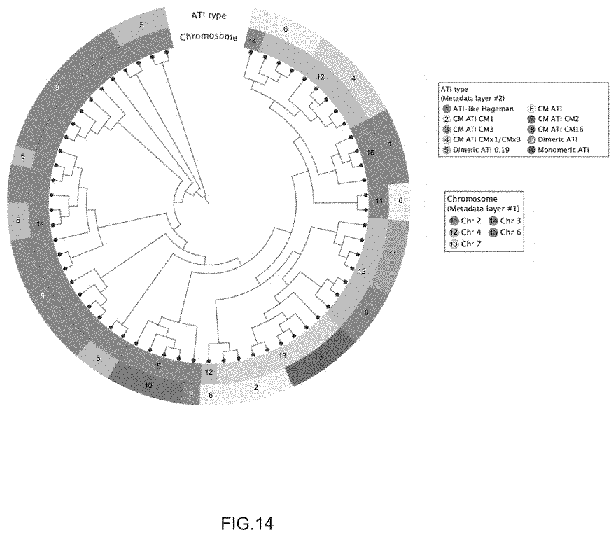

Two wheat cultivars, Baxter and Lancer, were used in the analysis as set out in Experiment 1. Altogether 265 proteins were identified in Baxter and 185 proteins in Lancer. From these proteins, 22 sequences corresponded with ATIs in Baxter and 19 ATIs were identified in Lancer. There were six shared ATI protein sequences detected from both genotypes. By comparing the protein sequences, three major ATI types (CM-ATIs, dimeric-ATIs and monomeric ATIs) were identified and differentiated 9 subtypes (including six sub-types of CM ATIs and two sub-types of dimeric ATIs) in both cultivars. When the sequences were compared to the wheat genome data dimeric ATIs, primarily known to be associated with Baker's asthma were mapped chromosome 3, while majority of the CM proteins, including CM3-ATIs that are associated with celiac disease were exclusively located on chromosome 4. Monomeric ATIs were mapped to chromosome 6 (FIG. 14).

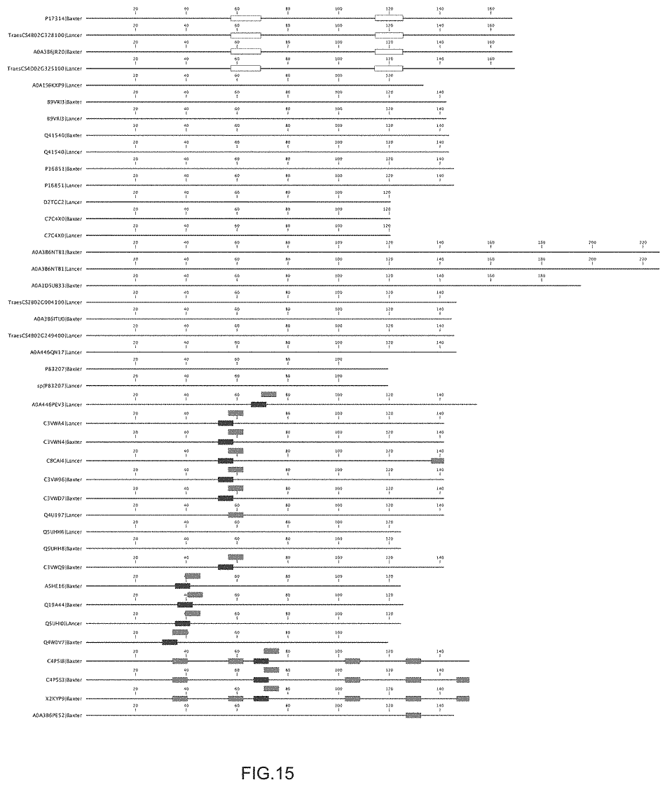

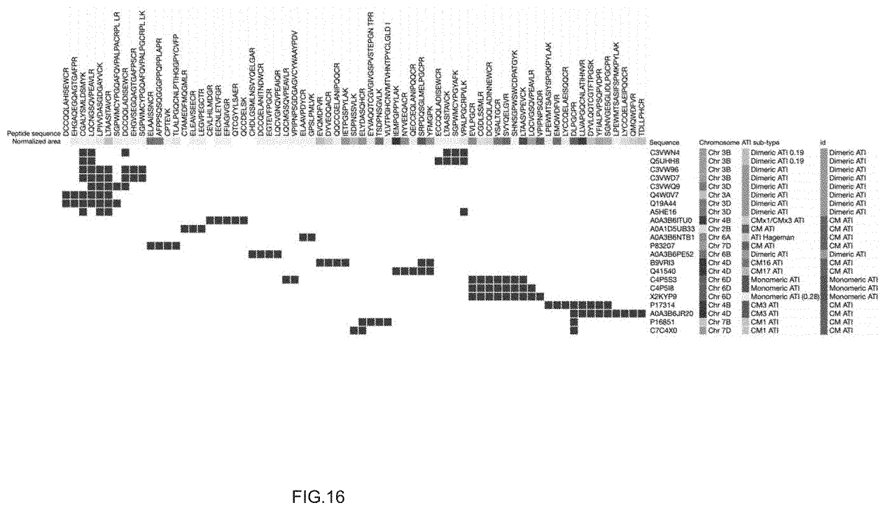

Epitope mapping analyses confirmed the presence of celiac disease associated TLR4 epitopes in the CM3 ATI sub-type and Baker's asthma-related B-cell epitopes in the dimeric and monomeric ATI sequences (FIG. 15). The IDA discovery analysis results and the identified ATI sequences were used for the targeted method developments. Altogether 69 ATI peptides identified from Baxter and Lancer were included in the analyses. Using peptide mapping with 100% sequence identity peptide sets specific for the following major ATI types: dimeric, 0.19 dimeric, monomeric, CM3, CM16-CM17 and CM1 ATIs (FIG. 16) were identified. Peptides overlapping with published celiac disease (Cuccioloni et al., 2017) or baker's asthma associated epitopes (Walsh 1998) were included in the analysis.

### Example 7: Wheat ATI Epitope Digestion by a Proteolytic Preparation Comprising Carica Papaya Endopeptidases

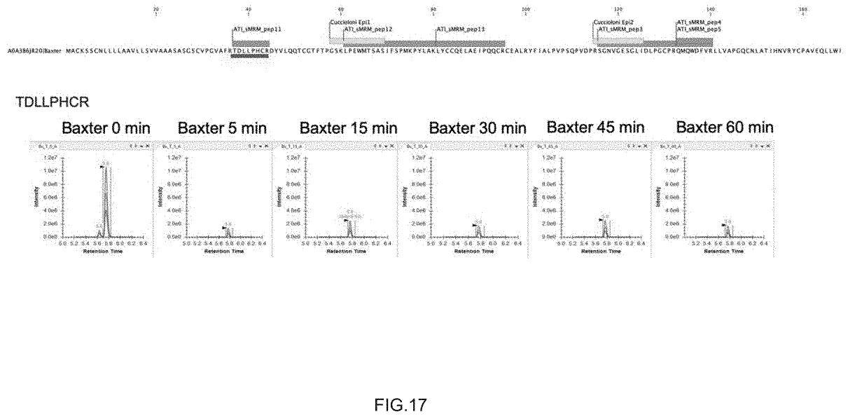

A targeted proteomics approach was used to quantify relative changes in fully tryptic or chymotryptic ATI peptide abundance after digestion with a proteolytic preparation comprising Carica papaya endopeptidases for 5, 15, 30, 45 and 60 minutes followed by an overnight digestion using trypsin or chymotrypsin. Peak intensity values were measured for each transition in Baxter and Lancer samples and peptide abundance values were calculated as a sum of the area under the curve values of the individual transitions (FIG. 17).

This data demonstrates the proteolytic preparation comprising Carica papaya endopeptidases (Enz 1) can efficiently cleave ATI epitopes. In particular, this data demonstrates the proteolytic preparation comprising Carica papaya endopeptidases (Enz 1) can efficiently cleave the epitope TDLLPHCR.

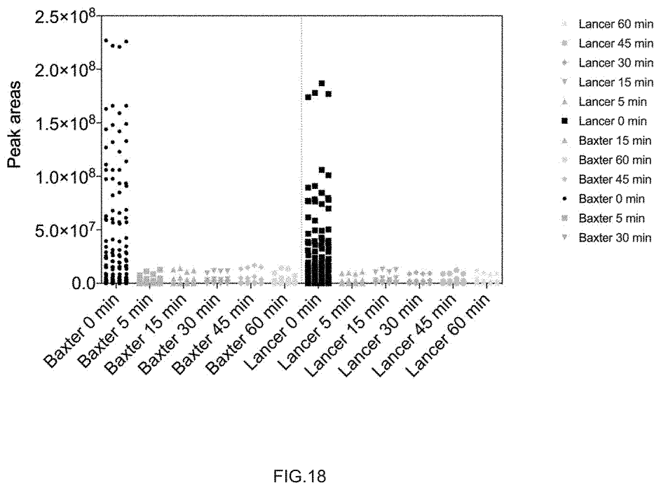

Initial peptide abundance levels measured at 0 minutes demonstrate a significant quantitative variation between the two cultivars resulting in peptide peak area values between 103 to 108. Peptides identified from monomeric ATIs, such as SVYQELGVR, SHNSGPWSWCDPATGYK or LQCVGSQVPEAVLR were detected in low amount in Lancer (peak area <10000), while the same peptides show 39,000×higher abundance in Baxter. An opposite trend has been seen in the dimeric ATI peptides, where higher peptide abundance values were detected in Lancer. When the overall ATI abundance levels were compared in the two cultivars across the time points between 0 to 60 minutes a significant decrease in abundance level has been detected after five minutes of digestion (FIG. 18). This data demonstrates the proteolytic preparation comprising Carica papaya endopeptidases (Enz 1) can efficiently cleave monomeric ATI epitopes. In particular, this data demonstrates the proteolytic preparation comprising Carica papaya endopeptidases (Enz 1) can efficiently cleave the epitopes SVYQELGVR, SHNSGPWSWCDPATGYK and LQCVGSQVPEAVLR.

Importantly, SHNSGPWSWCDPATGYK and LQCVGSQVPEAVLR which are cleaved, are associated with Baker's asthma.

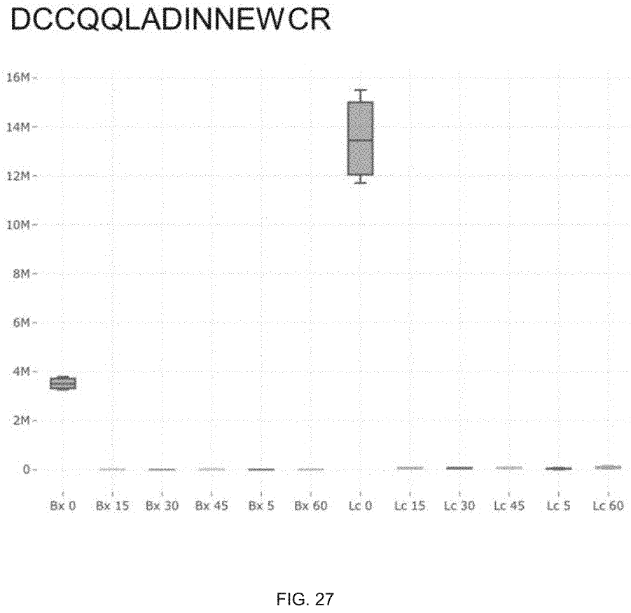

The present inventors have also demonstrated that the proteolytic preparation comprising Carica papaya endopeptidases (Enz 1) can efficiently cleave the epitope DCCQQLADINNEWCR (FIG. 27).

The cleavage of the epitopes EVLPGCR, CGDLSSMLR, VSALTGCR, LTAASVPEVCK, and VPIPNPSGDR is also examined.

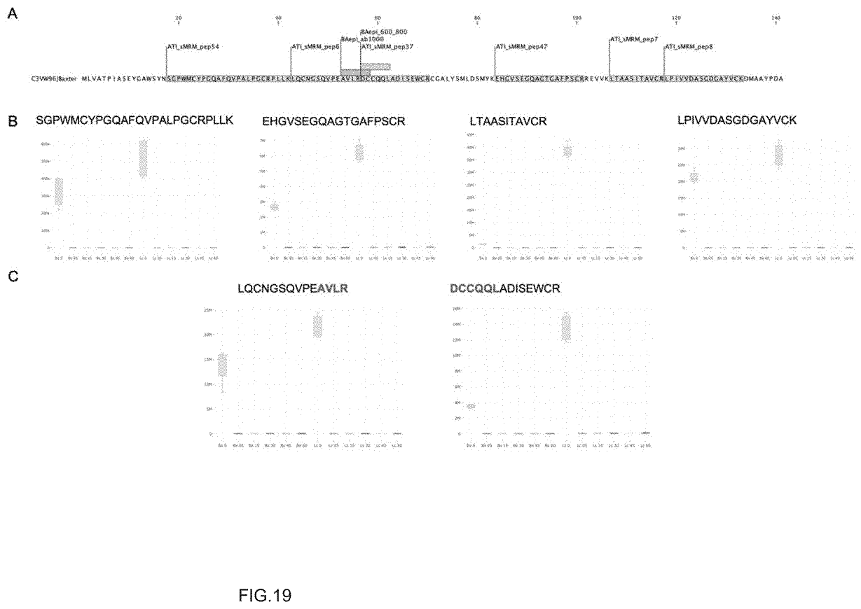

Altogether six dimeric ATI isoforms were detected in Lancer and eight isoforms in Baxter of which 1-1 sequence belong to the celiac disease associated dimeric-0.19 ATI subtype. The majority of the proteins contain epitopes associated with Baker's asthma (Welsh 1998). Identified protein sequences and measured peptide abundance values show a significant variation between the two cultivars (FIG. 19) indicating that dimeric ATIs are more abundant in Lancer. Digestion analysis confirms that all the peptides are cleaved after 5 minutes of digestion.

The 0.19 dimeric ATIs were represented by the isoforms Q5UHH6 and Q5UHH8. One peptide ECCQQLADISEWCR specific for this sub-type was identified, while the peptides SGPWMCYPGYAFK and LTAASITAVCK are shared between 0.19 and 0.53 dimeric ATIs. Interestingly, the ECCQQLADISEWCR peptide could only be measured in Baxter at 0 minutes but in Lancer, while the other two peptides were present in significantly higher amount in Lancer and cleaved after five minutes.

This data demonstrates the proteolytic preparation comprising Carica papaya endopeptidases (Enz 1) can efficiently cleave dimeric ATI epitopes. In particular, this data demonstrates the proteolytic preparation comprising Carica papaya endopeptidases (Enz 1) can efficiently cleave the epitopes SGPWMCYPGQAFQVPALPGCRPLLK, EHGVSEGQAGTGAFPSCR, LTAASITAVCR, LPIVVDASGDGAYVCK, LQCNGSQVPEAVLR, and DCCQQLAHISEWCR. This data also demonstrates the proteolytic preparation comprising Carica papaya endopeptidases (Enz 1) can efficiently cleave the epitopes ECCQQLADISEWCR, SGPWMCYPGYAFK, LTAASITAVCK and ECCQQLADISEWCR.

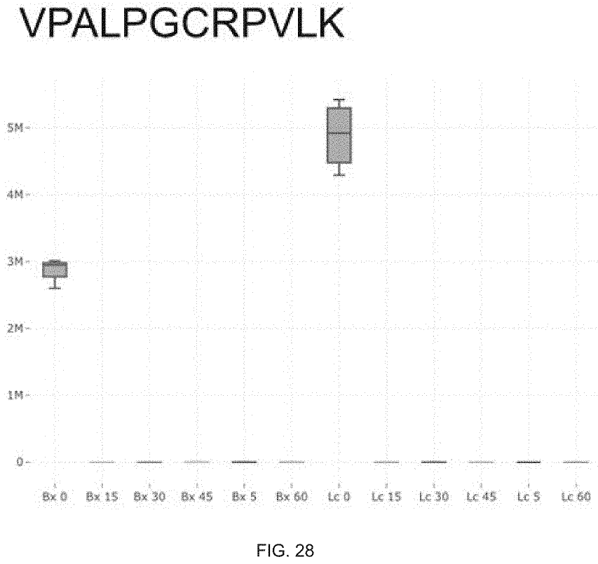

The present inventors have also demonstrated that the proteolytic preparation comprising Carica papaya endopeptidases (Enz 1) can efficiently cleave the epitope VPALPGCRPVLK (FIG. 28).

The cleavage of the epitopes DCCQQLAHISEWCR, EHGAQEGQAGTGAFPR, CGALYSMLDSMYK, SGPWMCYPGQAFQVPALPACRPLLR and VPALPGCRPVLK is also examined.

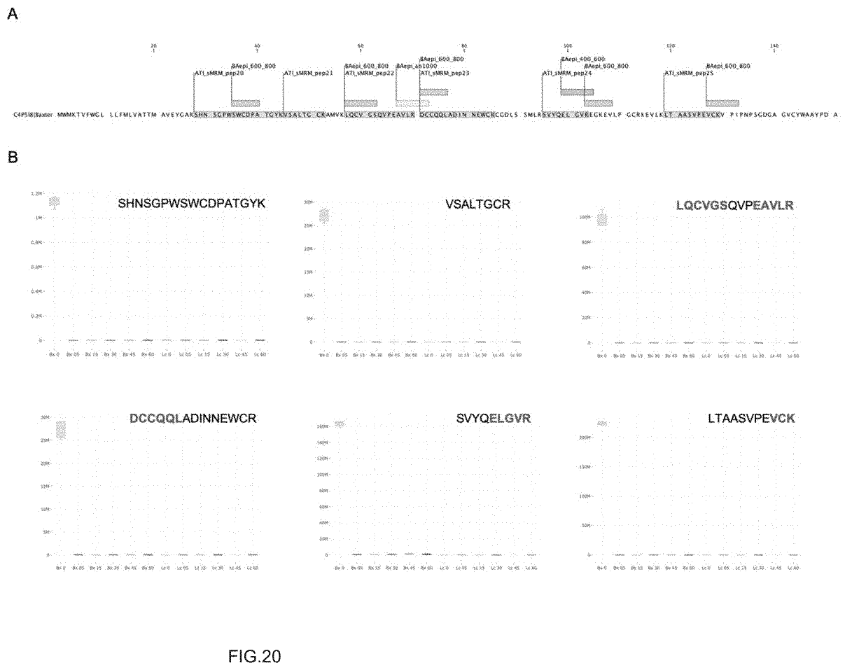

Monomeric ATIs are heavily enriched in peptide regions triggering immune response in Baker's asthma. In Baxter four isoforms of monomeric ATIs were identified, while there was no monomeric sub-type present in Lancer. All the monitored peptide sequences show a significant drop in peptide abundance level (FIG. 20). This data demonstrates the proteolytic preparation comprising Carica papaya endopeptidases (Enz 1) can efficiently cleave monomeric ATI epitopes. In particular, this data demonstrates the proteolytic preparation comprising Carica papaya endopeptidases (Enz 1) can efficiently cleave the epitopes SHNSGPWSWCDPATGYK, VSALTGCR, LQCVGSQVPEAVLR, DCCQQLADINNEWCR, SVYQELGVR, and LTAASVPEVCK. The present inventors have also demonstrated that the proteolytic preparation comprising Carica papaya endopeptidases (Enz 1) can efficiently cleave the epitope DCCQQLADINNEWCR (FIG. 27).

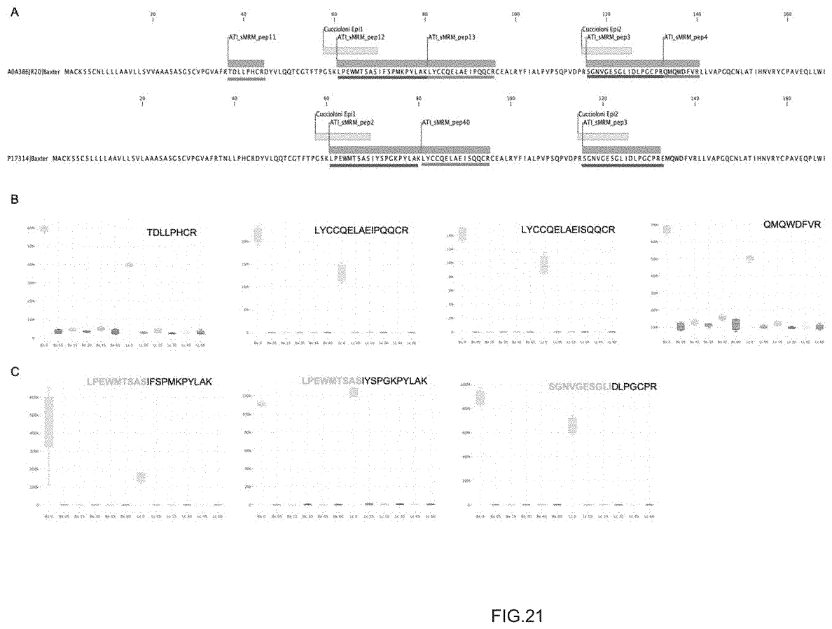

There were two CM3 ATI protein isoforms (UniProt accessions: P17314 and A0A3B6JR20) slightly differing in their protein sequences detected both in Baxter and Lancer. Both proteins include the TLR4 epitopes (Cuccioloni et al., 2017) and fully tryptic peptides overlapping with these epitopes have also been identified (FIG. 10A). While the A0A3B6JR20 isoform is present in higher abundance in Baxter, P17314 isoform was detected in slightly higher amount in Lancer. All the CM3 peptides show a significant decrease in their abundance 5 minutes after digestion. Monitoring the peptides overlapping with the epitopes confirm that the enzymes present in the proteolytic preparation comprising Carica papaya endopeptidases (Enz 1) can efficiently cleave these epitopes (FIG. 21C). This data demonstrates the proteolytic preparation comprising Carica papaya endopeptidases (Enz 1) can efficiently cleave CM3 ATI epitopes. In particular, this data demonstrates the proteolytic preparation comprising Carica papaya endopeptidases (Enz 1) can efficiently cleave the epitopes TDLLPHCR, LYCCQELAEISQQCR, LYCCQELAEIPQQCR, QMQWDFVR, and the TLR4 epitopes LPEWMTSASIYSPGKPYLAK, LPEWMTSASIFSPMKPYLAK, and SGNVGESGLIDLPGCPR, in particular LPEWMTSAS and SGNVGESGLI.

Cleavage of the epitopes EMQWDFVR, DLPGCPR, LLVAPGQCNLATIHNVR, DYVLQQTCGTFTPGSK, and YFIALPVPSQPVDPR is also examined.

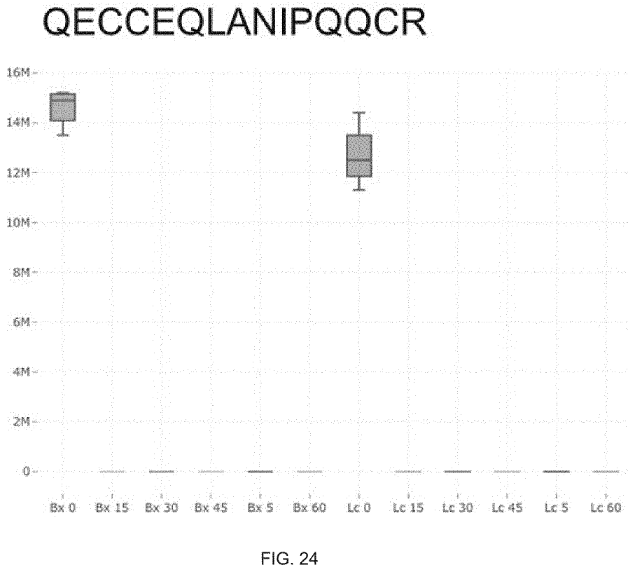

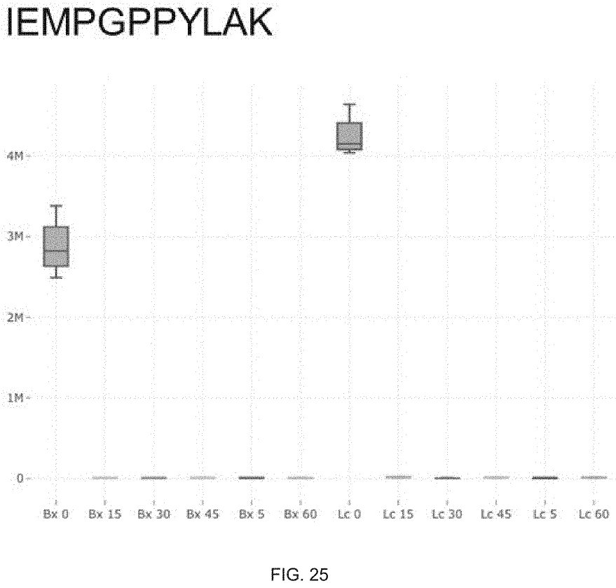

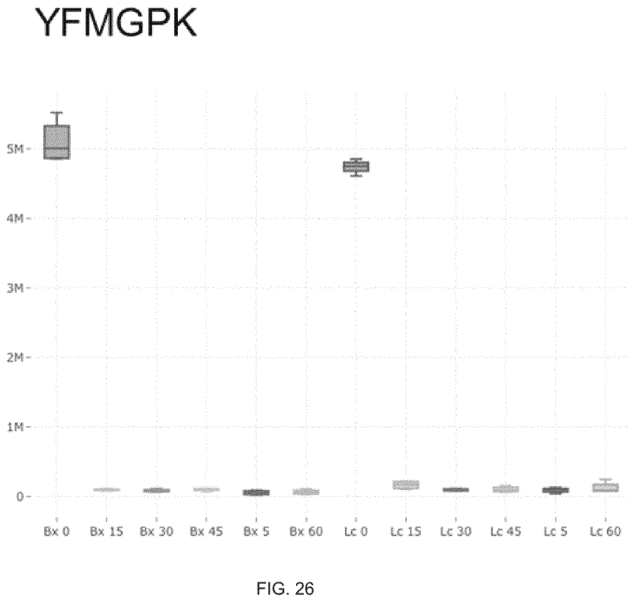

CM16 and CM17 ATI epitopes were examined. Monitoring the peptides overlapping with the epitopes confirm that the enzymes present in the proteolytic preparation comprising Carica papaya endopeptidases (Enz 1) can efficiently cleave the epitopes IEMPGPPYLAK (FIG. 25), QECCEQLANIPQQCR (FIG. 24), and YFMGPK (FIG. 26). This data demonstrates the proteolytic preparation comprising Carica papaya endopeptidases (Enz 1) can efficiently cleave CM3 ATI epitopes. In particular, this data demonstrates the proteolytic preparation comprising Carica papaya endopeptidases (Enz 1) can efficiently cleave the epitopes IEMPGPPYLAK (FIG. 25), QECCEQLANIPQQCR (FIG. 24), and YFMGPK (FIG. 26).

### Example 8: Digestion Specificity Analysis of the Proteolytic Preparation Comprising Carica Papaya Endopeptidases (Enz 1)

LC-MS/MS analysis focusing on the identification of proteins present in the proteolytic preparation comprising Carica papaya endopeptidases (Enz 1) indicate that at least four different enzymes are present, including caricain, multiple isoforms of papain, chymopapain and papaya proteinase 4. The present inventors have also demonstrated using mass spectrometry that a proteolytic preparation comprising one or more Carica papaya endopeptidases as described herein (Enz 1) also comprises Glutamine cyclotransferase (data not shown).

Peptide sequences detected in the F1 filtrates were used to determine the cleavage specificity of enzymes present in the proteolytic preparation comprising Carica papaya endopeptidases (Enz 1). As shown in FIG. 13, combinations of amino acid residues present in P3, P2, P1 and P1′, P2′, P3′ positions of the detected peptides were analysed first without filtering for ATI sequences, followed by an ATI-specific analysis. In case of the ATI-specific analysis, peptide sequences detected from the F1 filtrates were mapped to sequences with 100% sequence identity, and extended peptides, with 3 amino acid flanking regions in both directions were extracted and further analysed.

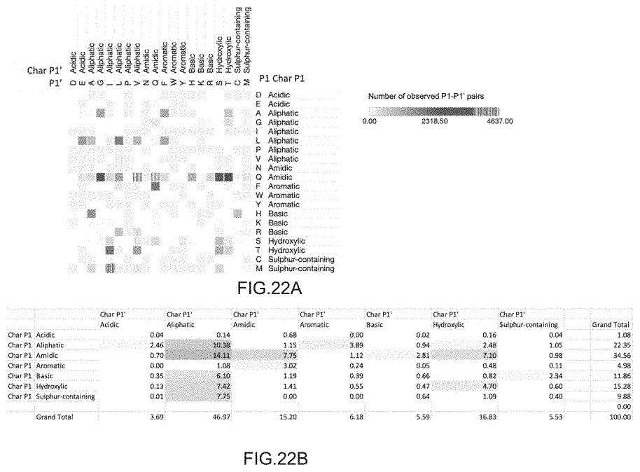

When the positions P1 and P1′ were identified for all the detected peptides that also includes peptides from other protein types such as gliadins, the most frequent P1-P1′ pairs were Q-Q; Q-V and M-I (FIG. 22A). These primarily represented an amidic residue in position P1 in combination with aliphatic, amidic or sulphur containing residue in position P1′ (FIG. 22B).

When the adjacent residues were also considered the most frequent combination was when the sequences were cleaved before an aliphatic amino acid in all P1′, P2′ and P3′ positions combined with cleavage after an aliphatic residue in P3, hydroxylic in P2 and Sulphur-containing in P1. Of these, the most frequent amino acid combination was P1′=I; P2′=A; P3′=L combined with P1=M; P2=T and P3=P.

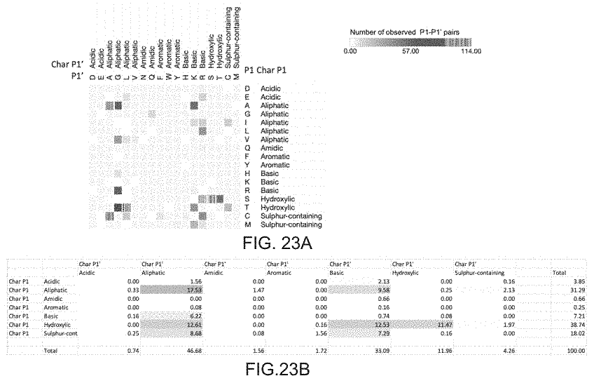

The ATI-specific patterns confirm the importance of aliphatic residues both in P1′ and P1 positions (FIG. 23B). Detailed analysis confirms that the most frequent amino acid residue pairs in P1 and P1′ positions are S and R, followed by S—S and A-A pairs (FIG. 23A). Based on the observed characteristic cleavage after a hydroxylic residue in combination with either an aliphatic, basic or hydroxylic residue explains more than 36% of the cleavage patterns.

### Example 9: Non-Reduced Wheat ATI Epitope Digestion by a Proteolytic Preparation Comprising Carica papaya Endopeptidases

The present inventors have demonstrated that ATIs can be enriched in a diluted ethanol extraction buffer primarily used to enrich gluten proteins. This sample preparation method avoids the use of reducing agent (DTT) and alkylation (using iodoacetamide) to mimic the conditions in the human digestive tract.

Accordingly, targeted analysis of α-amylase/trypsin inhibitor-specific tryptic peptides was performed to investigate the digestion efficiency of a proteolytic preparation comprising Carica papaya endopeptidases and An-PEP digestive supplements on gluten proteins in wheat. Briefly, proteins were extracted from 20 mg flour in four replicates using 70% ethanol. A proteolytic preparation comprising Carica papaya endopeptidase and An-PEP digestive supplement was prepared as described previously, and 20 μL of 0.1×solutions were used for the time course experiment in a final reaction volume of 200 μL resulting in a final supplement concentration of 0.01×. We performed four separate experiment to monitor the digestion efficiency of a proteolytic preparation comprising Carica papaya endopeptidases (Enz1) and An-PEP on ATIs, with and without the presence of pepsin. Conditions of the four experiments and data collection were as described above.

Monitored ATI-Specific Peptide Set

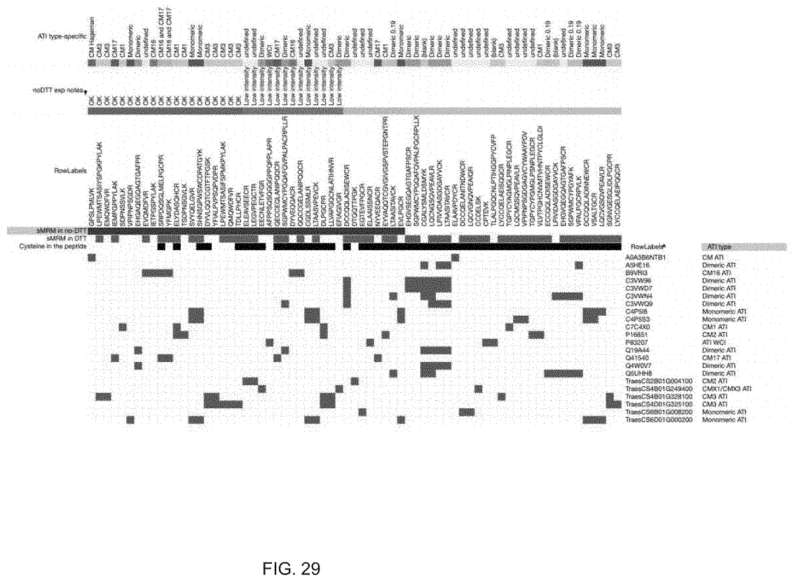

The same, fully tryptic peptide transitions were used to monitor the ATIs as described above. The list comprised of altogether 69 peptides characteristic to wheat cultivar Baxter of which 47 were reported in the previous ATI analysis as highly intense peptides detected in reducing conditions. In the no-DTT experiment there were 42 peptides detected of which 22 fully tryptic peptides were intense and were kept in the analysis. These peptides primarily represent the CM sub-class of ATIs (CM1, CM3, CM16 and CM17), while monomeric and dimeric ATI-specific peptides were under-represented (FIG. 29). Most of the peptides showing good intensities were those lacking cysteine residues in their sequence. Most of the cysteine containing peptides were barely detected, i.e., below the threshold intensities. As the protein digestion workflow did not incorporate reduction/alkylation steps, Cys-containing peptides may contain disulfide linkages which would preclude their detection/identification.

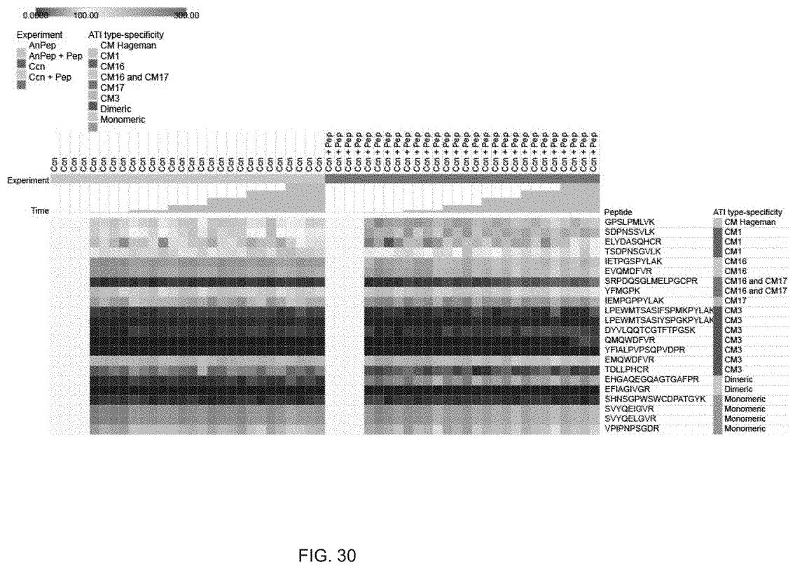

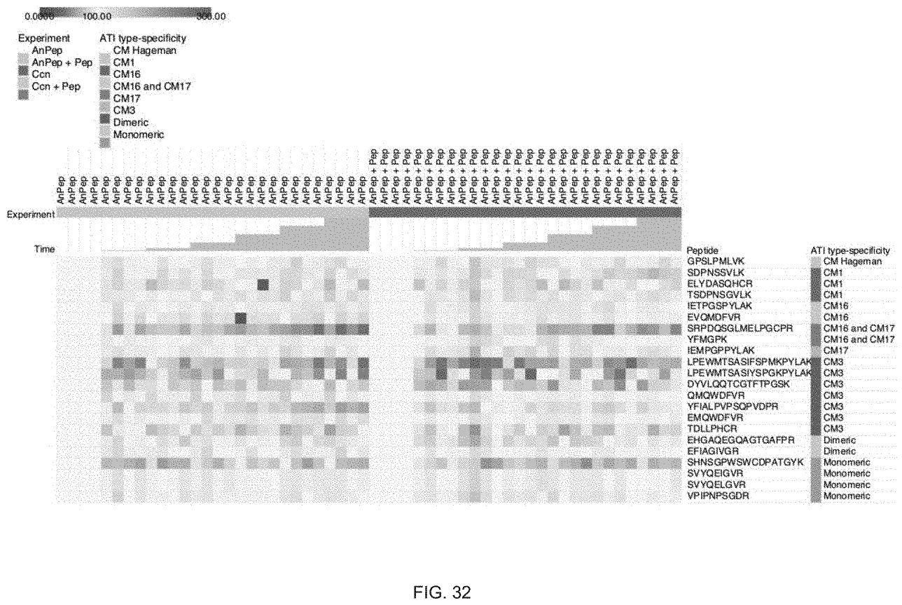

Peptide peak abundance values were normalized against the 0 time point samples in all four experiments (Ccn, Ccn+Pep, An-PEP and An-PEP+Pep) individually. The heatmaps below shows these 0 normalized abundance results, where 100% is labelled in pale grey, while decrease in relative abundance is labelled on a blue scale and increase is labelled in a red scale (FIG. 30—digestions using the proteolytic preparation comprising Carica papaya endopeptidases (Enz 1) and FIG. 32—An-PEP digestions).

FIG. 30 demonstrates the proteolytic preparation comprising Carica papaya endopeptidases (Enz 1) can efficiently cleave ATI epitopes in non-reducing conditions. In particular, this data demonstrates the proteolytic preparation comprising Carica papaya endopeptidases (Enz 1) can efficiently cleave the epitopes

GPSLPMLVK
SDPNSSVLK
IETPGSPYLAK
EVQMDFVR
SRPDQSGLMELPGCPR
YFMGPK
IEMPGPPYLAK
LPEWMTSASIFSPMKPYLAK
LPEWMTSASIYSPGKPYLAK
DYVLQQTCGTFTPGSK
QMQWDFVR
YFIALPVPSQPVDPR
EMQWDFVR
TDLLPHCR
EHGAQEGQAGTGAFPR
EFIAGIVGR
SHNSGPWSWCDPATGYK
SVYQEIGVR
SVYQELGVR
VPIPNPSGDR

All the CM16 peptides show a significant decrease in their abundance after digestion. Monitoring the peptides overlapping with the epitopes confirm that the enzymes present in the proteolytic preparation comprising Carica papaya endopeptidases (Enz 1) can efficiently cleave these epitopes in non-reducing conditions (FIG. 30). This data demonstrates the proteolytic preparation comprising Carica papaya endopeptidases (Enz 1) can efficiently cleave CM16 ATI epitopes in non-reducing conditions.

The CM17 peptide and the CM Hagerman peptide show a significant decrease in their abundance after digestion. Monitoring the peptides overlapping with the epitopes confirm that the enzymes present in the proteolytic preparation comprising Carica papaya endopeptidases (Enz 1) can efficiently cleave these epitopes in non-reducing conditions (FIG. 30). This data demonstrates the proteolytic preparation comprising Carica papaya endopeptidases (Enz 1) can efficiently cleave CM17 and CM Hagerman ATI epitopes in non-reducing conditions.

All the CM16 and CM17 peptides show a significant decrease in their abundance after digestion. Monitoring the peptides overlapping with the epitopes confirm that the enzymes present in the proteolytic preparation comprising Carica papaya endopeptidases (Enz 1) can efficiently cleave these epitopes in non-reducing conditions (FIG. 30). This data demonstrates the proteolytic preparation comprising Carica papaya endopeptidases (Enz 1) can efficiently cleave CM16 and CM17 ATI epitopes in non-reducing conditions.

All the CM3 peptides show a significant decrease in their abundance after digestion. Monitoring the peptides overlapping with the epitopes confirm that the enzymes present in the proteolytic preparation comprising Carica papaya endopeptidases (Enz 1) can efficiently cleave these epitopes in non-reducing conditions (FIG. 30). This data demonstrates the proteolytic preparation comprising Carica papaya endopeptidases (Enz 1) can efficiently cleave CM3 ATI epitopes in non-reducing conditions.

All the dimeric peptides show a significant decrease in their abundance after digestion. Monitoring the peptides overlapping with the epitopes confirm that the enzymes present in the proteolytic preparation comprising Carica papaya endopeptidases (Enz 1) can efficiently cleave these epitopes in non-reducing conditions (FIG. 30). This data demonstrates the proteolytic preparation comprising Carica papaya endopeptidases (Enz 1) can efficiently cleave dimeric ATI epitopes in non-reducing conditions.

All the monomeric peptides show a significant decrease in their abundance after digestion. Monitoring the peptides overlapping with the epitopes confirm that the enzymes present in the proteolytic preparation comprising Carica papaya endopeptidases (Enz 1) can efficiently cleave these epitopes in non-reducing conditions (FIG. 30). This data demonstrates the proteolytic preparation comprising Carica papaya endopeptidases (Enz 1) can efficiently cleave monomeric ATI epitopes in non-reducing conditions.

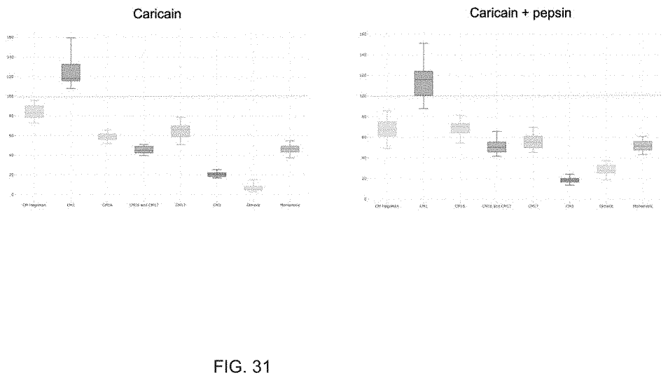

The mean of relative changes in peptide abundance was calculated for each ATI-specific peptide group separately (FIG. 31). The highest decrease in abundance (>80%) was measured in the Dimeric ATI and CM3-ATI peptides, while the CM16, CM17 and monomeric group specific peptides were only reduced by 40-50%. Presence of pepsin did not affect the peptide digestibility significantly.

FIG. 31 demonstrates the proteolytic preparation comprising Carica papaya endopeptidases (Enz 1) can efficiently cleave CM Hagerman, dimeric, monomeric, CM16, CM17, and CM3 ATI epitopes in non-reducing conditions.

### Example 10: Non-Reduced Wheat ATI Epitopes are not Digested by an-PEP; Aspergillus Niger-Derived Prolyl Endoprotease

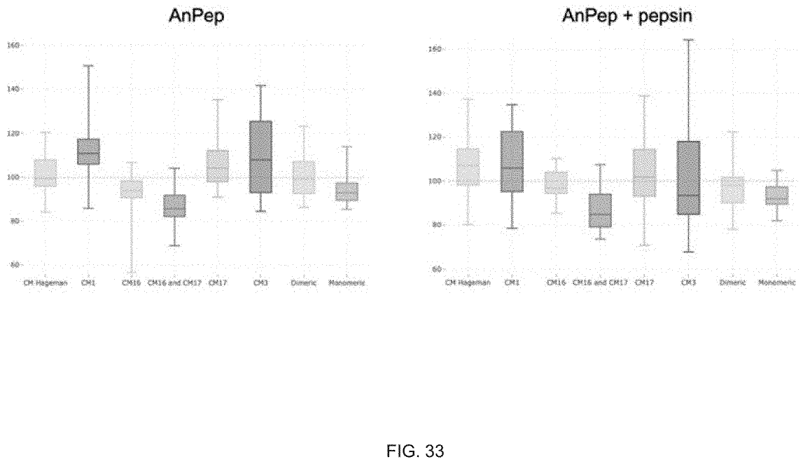

FIG. 32 shows digestion by An-PEP in presence and absence of pepsin confirmed that An-PEP is not able to digest ATIs effectively, and FIG. 33 shows relative changes of the group specific peptides showing An-PEP is not suitable to digest ATIs.

An-PEP is the Aspergillus niger-derived prolyl endoprotease (AN-PEP) has previously been shown to degrade gluten in healthy subjects when added to an intragastrically infused meal.

Filtrate 1 samples, representing peptides <10 kDa in size were collected form all four experiments as indicated earlier in the Gliadin experiment report. Unique Filtrate 1 peptides with confidence level >95% were used for the mapping analysis. Peptides were mapped with 100% sequence identity to the ATI proteins identified in Baxter. Interestingly, only two peptides (QPVDPRSGNVGESGL and VEYGARSH), characteristic on CM3 and monomeric ATIs could be detected from the Filtrate 1 60 min samples. While VEYGARSH is located at the N-terminal end of the proteins, QPVDPRSGNVGESGL is positioned toward the C-terminus of the proteins. Both peptides overlap with fully tryptic peptides monitored in this analysis, which indicates the variable nature of digestion of ATIs by caricain, i.e., lower digestion specificity compared to trypsin.

Examples 9 and 10 demonstrate that while the proteolytic preparation comprising Carica papaya endopeptidases (Enz 1) is also able to digest the various ATI proteins under non-reducing conditions, An-PEP is not as effective on ATIs. The presence of pepsin does not significantly affect the digestibility. The differences in the detection of high intensity peptides in reducing and non-reducing conditions indicate that protein regions buried due to the compact globular structure of ATIs fixed by internal disulfide bridges are less prone to caricain digestion.

What is claimed:

1. A method for digesting at least one alpha amylase trypsin inhibitor (ATI), the method comprising contacting an ATI with an effective dose of a composition comprising a proteolytic preparation comprising one or more Carica papaya endopeptidases.

2. A method for digesting at least one alpha amylase trypsin inhibitor (ATI) in an ATI-containing foodstuff, the method comprises contacting the ATI-containing foodstuff with an effective dose of a composition comprising a proteolytic preparation comprising one or more Carica papaya endopeptidases.

3. A method according to claim 1 wherein the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5).

4. A method according to claim 1 wherein the alpha amylase trypsin inhibitor (ATI), is contacted with the proteolytic preparation comprising one or more Carica papaya endopeptidases under conditions sufficient to cause digestion of the ATI by hydrolysis.

5. A method according to claim 1 wherein the at least one ATI is an ATI derived from a wheat, barley, rye or oat species, preferably wherein the at least one ATI is an ATI derived from a spelt, khorasan, emmer, einkorn, and triticale species.

6. (canceled)

7. (canceled)

8. A method according to claim 1 wherein at least one epitope of the at least one ATI is digested, preferably wherein the at least one epitope is selected from;
a) an Alpha-amylase inhibitor precursor (CIII) (WMAI-1) B cell epitope selected from the group consisting of:
AASVPE
ADINNE
ALTGCR
AMVKLQ
AVLRDC
AYPDV
CQQLAD
CRAMVK
CVGSQV
CYGDWA
DCCQQL
ELGVRE
EVMKLT
GCRKEV
GDRAGV
GDWAAY
GKEVLP
GSQVPE
GVCYGD
GVREGK
INNEWC
KVPIPN
LPGCRK
LQCVGS
LRDCCQ
LRSVYQ
LSSMLR
LTAASV
MKLTAA
NEWCRC
PATGYK
PEAVLR
PEVCKV
PSGDRA
QLADIN
QVPEAV
RAGVCY
RCGDLS
REGKEV
RKEVMK
SGPWSW
SMLRSV
SVPEVC
SVYQEL
TGCRAM
TGYKVS
VCKVPI
VKLQCV
VSALTG
WAAYPD
WCRCGD
YKVSAL
YQELGV;
b) a putative alpha-amylase inhibitor B cell epitope selected from the group consisting of PWSWCD, SWCDPA, and SWCDPATGYKVSALTGCRAMV;
c) a peptide comprising an ATI epitope, said peptide selected from the group consisting of:
DCCQQLAHISEWCR
EHGAQEGQAGTGAFPR
CGALYSMLDSMYK
LQCNGSQVPEAVLR
LPIVVDASGDGAYVCK
LTAASITAVCR
SGPWMCYPGQAFQVPALPACRPL LR
DCCQQLADISEWCR
EHGVSEGQAGTGAFPSCR
SGPWMCYPGQAFQVPALPGCRPL LK
ECCQQLADISEWCR
LTAASITAVCK
SGPWMCYPGYAFK
VPALPGCRPVLK:
d) a CM16 and/or CM17 ATI epitope selected from the group consisting of:
IETPGSPYLAK
SDPNSSVLK
ELYDASQHCR
EYVAQQTCGVGIVGSPVSTEPGN TPR
TSDPNSGVLK
VLVTPGHCNVMTVHNTPYCLGLD I
IEMPGPPYLAK
NYVEEQACR
QECCEQLANIPQQCR
SRPDQSGLMELPGCPR
YFMGPK
EVQMDFVR:
e) a monomeric ATI epitope selected from the group consisting of:
EVLPGCR
CGDLSSMLR
DCCQQLADINNEWCR
VSALTGCR
SVYQELGVR
SHNSGPWSWCDPATGYK
LTAASVPEVCK
LQCVGSQVPEAVLR
VPIPNPSGDR
SVYQEIGVR;
f) a CM3 ATI epitope selected from the group consisting of:
LPEWMTSASIYSPGKPYLAK
EMQWDFVR
LYCCQELAEISQQCR
DLPGCPR
LLVAPGQCNLATIHNVR
DYVLQQTCGTFTPGSK
YFIALPVPSQPVDPR
SGNVGESGLIDLPGCPR
LPEWMTSASIFSPMKPYLAK
LYCCQELAEIPQQCR
QMQWDFVR
TDLLPHCR;
g) a CM Hagerman or CM1 ATI epitope selected from the group consisting of:
GPSLPMLVK
SDPNSSVLK;
and
h) a dimeric ATI epitope selected from the group consisting of:
EHGAQEGQAGTGAFPR
EFIAGIVGR.

9. (canceled)

10. A method according to claim 1 wherein the proteolytic preparation is derived from Carica papaya oleoresin or Carica papaya latex.

11. (canceled)

12. A method according to claim 1 wherein the composition further comprises one or more excipients.

13. A method according to claim 1 wherein the method is performed in vitro or in vivo.

14. A method according to any one of claims 1 to 12 wherein the composition is administered to a human.

15. A method for the treatment and/or prevention of an alpha-amylase/trypsin inhibitor (ATI) mediated condition, comprising administering to a subject in need thereof an effective amount of a composition comprising a proteolytic preparation comprising one or more Carica papaya endopeptidases, preferably wherein the condition is a wheat alpha-amylase/trypsin inhibitor (ATI) mediated condition.

16. (canceled)

17. A method according to claim 15 wherein the alpha-amylase/trypsin inhibitor (ATI) mediated condition is wheat allergy, non-celiac gluten/wheat sensitivity, irritable bowel syndrome or baker's asthma.

18. A method according to claim 15 wherein the proteolytic preparation comprises one or more protease selected from the group consisting of papain (EC 3.4.22.2), caricain (EC 3.4.22.30), chymopapain (EC 3.4.22.6), Papaya proteinase 4 (EC 3.4.22.25), and glutamine cyclotransferase (EC 2.3.2.5).

19. A method according to claim 15 to 18 wherein the proteolytic preparation comprises is derived from Carica papaya oleoresin.

20. A method according to claim 15 to 19 wherein the proteolytic preparation comprises is derived from Carica papaya latex.

21. A method according to claim 15 to 20 wherein the composition further comprises one or more pharmaceutically acceptable excipients.

22. A method according to claim 15 to 21 wherein the composition is administered orally.

23. A method according to claim 15 to 22, wherein the composition is enterically coated.

24. A method according to claim 15 to 23 wherein the subject is a human subject.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)
